SEQUENCE LISTING (37 sequences)

SEQ ID NO: 1 - protein, 21 aa, synthetic construct
NKKFLPFQQFGRDIADTTDAV

SEQ ID NO: 2 - protein, 22 aa, synthetic construct
NATYYNSWQNLLYDSNGNLYGF

SEQ ID NO: 3 - protein, 21 aa, synthetic construct
TEVPVAIHADQLTPTWRVYST

SEQ ID NO: 4 - protein, 15 aa, synthetic construct
NYVFNNSLTRQLQPI

SEQ ID NO: 5 - protein, 15 aa, synthetic construct
ASYQTQTNSPRRARS

SEQ ID NO: 6 - protein, 11 aa, synthetic construct
VDYSKNRRSRG

SEQ ID NO: 7 - protein, 19 aa, synthetic construct
ILPDPSKPSKRSFIEDLLF

SEQ ID NO: 8 - protein, 23 aa, synthetic construct
VLGCLGSECSKASSRSAIEDLLF

SEQ ID NO: 9 - protein, 27 aa, synthetic construct
PELDSFKEELDKYFKNHTSPDVDLGDI

SEQ ID NO: 10 - protein, 26 aa, synthetic construct
PNLPDFKEELDQWFKNQTSVAPDLSL

SEQ ID NO: 11 - protein, 35 aa, synthetic construct
IQKEIDRLNEVAKNLNESLIDLQELGKYEQYIKWP

SEQ ID NO: 12 - protein, 35 aa, synthetic construct
LQVEMNRLQEAIKVLNQSYINLKDIGTYEYYVKWP

SEQ ID NO: 13 - protein, 223 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 14 - protein, 21 aa, synthetic construct
NKKFLPFQQFGRDIADTTDAV

SEQ ID NO: 15 - protein, 21 aa, synthetic construct
TEVPVAIHADQLTPTWRVYST

SEQ ID NO: 16 - protein, 16 aa, synthetic construct
ASYQTQTNSPRRARSV

SEQ ID NO: 17 - protein, 19 aa, synthetic construct
ILPDPSKPSKRSFIEDLLF

SEQ ID NO: 18 - protein, 25 aa, synthetic construct
PELDSFKEELDKYFKNHTSPDVDLG

SEQ ID NO: 19 - protein, 35 aa, synthetic construct; identical to SEQ ID NO: 11

SEQ ID NO: 20 - protein, 196 aa, synthetic construct
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPK

SEQ ID NO: 21 - protein, 242 aa, synthetic construct
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSGGLNDIFEAQKIEWHEGSGEDQVDPRLIDGKGSGHHHHHHHHHH

SEQ ID NO: 22 - protein, 21 aa, synthetic construct
TEVPVAIHADQLTPTWRVYST

SEQ ID NO: 23 - protein, 31 aa, synthetic construct
GSGSTEVPVAIHADQLTPTWRVYSTGSGSGS

SEQ ID NO: 24 - protein, 16 aa, synthetic construct
ASYQTQTNSPRRARSV

SEQ ID NO: 25 - protein, 20 aa, synthetic construct
GSGSASYQTQTNSPRRARSV

SEQ ID NO: 26 - protein, 35 aa, synthetic construct; identical to SEQ ID NO: 11

SEQ ID NO: 27 - protein, 39 aa, synthetic construct
GSGSIQKEIDRLNEVAKNLNESLIDLQELGKYEQYIKWP

SEQ ID NO: 28 - protein, 21 aa, synthetic construct
NKKFLPFQQFGRDIADTTDAV

SEQ ID NO: 29 - protein, 24 aa, synthetic construct
GSGSNKKFLPFQQFGRDIADTTDA

SEQ ID NO: 30 - protein, 19 aa, synthetic construct
ILPDPSKPSKRSFIEDLLF

SEQ ID NO: 31 - protein, 23 aa, synthetic construct
GSGSILPDPSKPSKRSFIEDLLF

SEQ ID NO: 32 - protein, 25 aa, synthetic construct
PELDSFKEELDKYFKNHTSPDVDLG

SEQ ID NO: 33 - protein, 29 aa, synthetic construct
GSGSPELDSFKEELDKYFKNHTSPDVDLG

SEQ ID NO: 34 - protein, 4 aa, synthetic construct
GSGS

SEQ ID NO: 35 - protein, 15 aa, synthetic construct
  SITE 15: note = C-terminally modified with avitag
GLNDIFEAQKIEWHE

SEQ ID NO: 36 - protein, 12 aa, synthetic construct
EDQVDPRLIDGK

SEQ ID NO: 37 - protein, 10 aa, synthetic construct
HHHHHHHHHH

(19) United States
(12) Patent Application Publication
(10) Pub. No.: US 2024/0385189 A1
(43) Pub. Date: Nov. 21, 2024

(54) COMPOSITIONS AND METHODS FOR DETERMINING HUMORAL IMMUNE RESPONSES AGAINST SEASONAL CORONAVIRUSES AND PREDICTING EFFICIENCY OF SARS-COV-2 SPIKE TARGETING, COVID-19 DISEASE SEVERITY, AND PROVIDING INTERVENTIONS

(71) Applicant: Jacobs Technion-Cornell Institute, New York, NY (US)

(72) Inventors: Raymond ALVAREZ, New York, NY (US); Rebecca BRACHMAN, New York, NY (US)

(21) Appl. No.: 18/689,814

(22) PCT Filed: Sep. 9, 2022
(86) PCT No.: PCT/US2022/076228

§ 371 Date: Mar. 6, 2024

Related U.S. Application Data
(60) Provisional application No. 63/243,064, filed on Sep. 10, 2021.

(51) Int. Cl. G01N 33/569; G01N 33/543

(52) U.S. Cl. CPC G01N 33/56983; G01N 33/54386; G01N 2333/165; G01N 2469/20

(57) ABSTRACT

Provided are compositions and methods for use in determining antibody profiles from individuals who have been infected, vaccinated, or both infected and vaccinated with a one or more types of coronavirus. The compositions and methods can be used for predicting severity of outcomes, or for developing and implementing medical interventions.

### CROSS REFERENCE TO RELATED APPLICATION

This application claims priority to U.S. provisional application No. 63/243,064, filed Sep. 10, 2021, the entire disclosure of which is incorporated herein by reference.

### SEQUENCE LISTING

The instant application contains a Sequence Listing which has been submitted in .xml format and is hereby incorporated by reference in its entirety. Said .xml copy was created on Sep. 8, 2022, is named “095662_00002_ST26_XML”, and is 34,721 bytes in size.

### BACKGROUND

During the first waves of the coronavirus disease 2019 (COVID-19) pandemic, approximately 80% of individuals experienced asymptomatic or mild disease, 15% experienced moderately severe pneumonia, and 5% required hospitalization with severe acute respiratory distress syndrome (ARDS) (Wu and McGoogan, 2020). Studies conducted in patients with severe COVID-19 commonly observed a hyperimmune activation state (Del Valle et al., 2020; Tang et al., 2020; Ye et al., 2020), which coincided with the onset of adaptive immunity. In particular, several studies correlated early detection and higher anti-spike protein immunoglobulin G (IgG) titers with more severe disease (Long et al., 2020; Qu et al., 2020; Young et al., 2020; Zhang et al., 2020; Zhao et al., 2020), suggesting that humoral immunity may exacerbate COVID-19. Altogether, these findings have led several researchers to hypothesize that adaptive antibody (Ab) immunity may exacerbate disease severity through Ab-dependent enhancement (ADE) of disease (Iwasaki and Yang, 2020; Larsen et al., 2020; Lee et al., 2020; Ricke, 2021).

As a category, ADE refers to the processes by which pathogen-specific Abs increase virus replication (Ab-dependent enhancement of infection) and/or proinflammatory mediators (Ab-dependent immune enhancement [ADI]), both of which can enhance the severity of disease (Iwasaki and Yang, 2020). The effects of ADE have been observed in the context of several viral infections, including dengue, respiratory syncytial virus (RSV), and even other coronaviruses (Bournazos et al., 2020; Halstead, 2014; Smatti et al., 2018). Studies examining dengue-induced ADE have identified IgG-FcγR interactions as one of the primary factors governing increased disease severity (Mohsin et al., 2015; Thulin et al., 2020; Wang et al., 2017). Fcγ receptors (FcγRs) are a family of IgG-binding receptors that trigger a diverse array of non-neutralizing effector functions, such as Ab-dependent cellular cytotoxicity (ADCC), Ab-dependent cellular phagocytosis (ADCP), dendritic cell (DC) maturation and antigen presentation, and effector cytokine production (Forthal and Moog, 2009; Pincetic et al., 2014; Vogelpoel et al., 2015). Though FcγR activation is of critical importance in controlling viral infections in vivo, in the context of pathogens that induce ADE, high IgG-mediated FcγR activation has been shown to be detrimental. Notably, several studies have reported ADE/ADI effects during severe acute respiratory syndrome coronavirus 1 (SARS-CoV-1) infections in vitro and in vivo (Jaume et al., 2011; Liu et al., 2019; Wang et al., 2014; Yip et al., 2014). In contrast, vaccine studies in hamsters demonstrated that despite FcγR-IgG-mediated cellular uptake of virus, vaccinated animals were protected in viral challenge experiments (Kam et al., 2007). Therefore, the contribution of FcγR-IgG in the resolution of severe coronavirus infection remains unclear.

In most instances of virus-induced ADE, immunological memory responses from a previous infection become activated and mount inefficient and detrimental responses to a similar but distinct heterologous strain. While SARS-CoV-2 has now evolved variant strains that could act like heterologous strains, these strains arose only several months after the initial outbreak and cannot account for any ADE-like effects observed early in the pandemic. However, seasonal human coronaviruses (shCoVs), which are ubiquitous (Gorse et al., 2010), share some regions of high sequence identity with the SARS-CoV-2 spike protein, the primary viral antigen targeted by neutralizing Abs. The S2 subunit of the SARS-CoV-2 spike contains regions of high sequence conservation with shCoVs. In contrast, the receptor-binding domain (RBD)-containing S1 subunit, which interacts with host angiotensin-converting enzyme 2 (ACE2) receptor, bears far less sequence similarity to shCoVs. Studies have shown that Abs targeting the RBD region mediate potent neutralization of SARS-CoV-2 in vitro and in vivo (Rogers et al., 2020; Wang et al., 2020b; Wec et al., 2020; Zost et al., 2020). Pre-pandemic and SARS-CoV-2-naive samples possess IgG directed against the S2, but not the S1, subunit of the spike protein (Anderson et al., 2021; Ng et al., 2020; Nguyen-Contant et al., 2020). Notably, these sera are non-neutralizing and display high reactivity against the shCoV, OC43 (Anderson et al., 2021). In addition, other studies have observed that cross-reactive Ab responses against OC43 are elevated after SARS-CoV-2 infections (Shrock et al., 2020; Wang et al., 2020c). However, the contribution of shCoV cross-reactive IgG to the humoral response against SARS-CoV-2 remains unclear, with different studies showing both positive and negative correlations with severity (Anderson et al., 2021; Shrock et al., 2020; Wang et al., 2020c).

Beyond the characterization of IgG S1- versus S2-binding, other studies have conducted peptide walks to identify the linear epitopes targeted by humoral responses against the spike protein. These studies have identified several immunodominant regions that overlap or flank several functional features, such as the S1/S2 furin cleavage site (S1/S2), the S2′ fusion peptide region (S2′FP), and the heptad repeat (HR) 1 region and HR2 sites (Li et al., 2021; Mishra et al., 2021; Wang et al., 2020a; Zamecnik et al., 2020). Some of these immunodominant regions possess high sequence identity with shCoVs (Ladner et al., 2021; Shrock et al., 2020). These findings—coupled with IgG recognition of the S2 region in naive individuals and the elevated levels of OC43 cross-reactive IgG in convalescent individuals—suggest that preexisting recall responses against shCoVs likely contribute to the humoral response against SARS-CoV-2. However, the contribution of these cross-reactive Abs to the evolution of effective humoral immunity, disease severity, and outcomes remains unclear. Thus, there is an ongoing and unmet need for improved compositions and methods to address differences in immune responses to coronavirus infections. The disclosure is pertinent to this need.

### BRIEF SUMMARY

The present disclosure is related in part to the discovery that anti-spike IgG early in SARS-CoV-2 infection may be attributable to the amplification of humoral memory responses against seasonal human coronavirus (hCoVs) in severe COVID-19 patients. This disclosure provides characterization of anti-spike IgG from a cohort of non-hospitalized convalescent individuals with a spectrum of COVID-19 severity, as well as a cohort of ICU-hospitalized individuals with acute, severe COVID-19. The results demonstrate that anti-spike IgG levels positively correlated with disease severity, higher IgG cross-reactivity against betacoronaviruses (SARS-CoV-1 and OC43), and higher levels of proinflammatory Fc gamma receptor 2a and 3a (FcγR2a & FcγR3a) activation. In examining the levels of IgG targeting betacoronavirus cross-reactive epitopes and disease severity, we observed a positive correlation with the levels of IgG targeting the S2′FP region, and an inverse correlation with the levels of IgG targeting two epitopes around the heptad repeat (HR) 2 region. In comparing the levels of IgG targeting non-conserved epitopes, we observed that only one of three non-conserved immunodominant epitopes correlated with disease severity. As described in greater detail below, the levels of IgG targeting the RBD region were inversely correlated with severity. Targeting of the RBD and HR2 regions have both been shown to mediate SARS CoV-2 neutralization. The disclosure thus demonstrates that, aside from Ab targeting of the RBD region, humoral memory responses against seasonal betacoronaviruses are an important factor in dictating COVID-19 severity, with anti-HR2-dominant Ab profiles representing protective memory responses, while an anti-S2′FP dominant Ab profiles indicating deleterious recall responses. Though these profiles are masked in whole antigen profiling, data presented in this disclosure indicate that distinct Ab memory responses are detectable with epitope targeting analysis. In this regard, the description below expands on these discoveries to provide improved predictive approaches that involve determining ratios of antibodies that bind to particular combinations of peptides, including but not limited to composite antibody profile ratios. The disclosure thus provides compositions and methods for predicting severity of SARS-CoV-2 infections (primary and reinfections), and for use in predicting vaccine efficacy in individuals with different dominant antibody epitope profiles, as well as tailoring individual treatment recommendations and treatments based on the antibody profiles.

### BRIEF DESCRIPTION OF THE FIGURES

FIG. 1 panels show anti-spike IgG levels in convalescent donors correlate with COVID-19 severity. FIG. 1, panel (A). Anti-spike IgG titers in SARS-CoV-2 convalescent (CoV-2⁺) and naive (CoV-2⁻) donors, as quantified by ELISA (n=28 and 20, respectively). FIG. 1, panel (B). Levels of anti-spike IgG in convalescent (CoV-2⁺) donors, as quantified by ELISA. Horizontal line represents 3-fold above the mean anti-spike levels of naive (CoV-2⁻) donors, as quantified by ELISA. FIG. 1, panel (C). Comparison of ELISA-based anti-spike IgG titers versus CoV-2⁺ composite severity scores. FIG. 1, panels (D)-(F). Levels of anti-spike IgG in naive (CoV-2⁻) and CoV-2⁺ donors, as quantified using a cell-based assay (CBA). FIG. 1, panel (D). Levels of anti-spike IgG titers in CoV-2⁻ and CoV-2⁺ donors. FIG. 1, panel (E). Levels of anti-spike titers in CoV-2⁺ donors, as quantified by CBA. FIG. 1, panel (F). Comparison of CBA anti-spike IgG titers versus CoV-2⁺ composite severity scores. The SEMs of N=3 experiments are shown.

FIG. 2 panels show FcγR activation correlates with COVID-19 severity and anti-spike titers. FIG. 2 though its panels depicts the levels of FcγR-signaling induced by purified IgG from CoV-2⁺ donors in response to SARS-CoV-2 spike protein expressed on the surface of 293T cells. FIG. 2 panels A-D Graphs show the levels of (panel A and B) FcγRIIa and (panel C and D) FcγRIIIa signaling using 25 μg/mL IgG by (B and D) composite symptom severity scores or by (A and C) CoV−/+status and severity scores. FIG. 2 panels E-H. The panels compare the anti-spike IgG titers as quantified by CBA or ELISA assays versus the levels of FcγR signaling. (FIG. 2 panels E and F) CBA anti-spike IgG versus (FIG. 2 panel E) FcγRIIa and (F) FcγRIIIa signaling using 25 μg/mL IgG. (FIG. 2 panels G and H) ELISA anti-spike IgG titers versus (FIG. 2 panel G) FcγRIIa and (FIG. 2 panel H) FcγRIIIa signaling using 25 μg/mL IgG. All of the FcγR results are the SEMs of N=3 experiments.

FIG. 3 panels show higher β-coronavirus cross-reactive IgG titers are correlated with COVID-19 severity. FIG. 3 panels A, B, C, D, E, F, G, and H. Graphs compare the level of IgG binding to spike proteins of (panel A and panel E) SARS1, (panel B and panel F) OC43, (panel C and panel G) NL63, and (panel D and panel H) 229E coronavirus as assessed by CBA and detected by flow cytometry. Graphs (panels A, B, and C and D) compare the level of anti-spike IgG in SARS-CoV-2-naive versus-convalescent donors. Graphs (panel E, panel F, panel G and panel H) compare the levels of anti-spike IgG among donor groups separated by SARS-CoV-2 status and COVID-19 severity scores, n=13 mild, 15 severe. The SEMs of N=3 experiments are shown.

FIG. 4 panels show localization of SARS-CoV-2 spike immunodominant regions. FIG. 4 panel A. Diagram depicts SARS-CoV-2 spike protein subdomains, which include the N-terminal domain (NTD), the receptor-binding domain (RBD), S1-C terminus domains 1 and 2 (CTD1 and -2), the furin cleavage site (S1/S2); the S2′ cleavage site and fusion protein domain (S2′FP), the heptad repeat domains 1 and 2 (HR1 and -2), the transmembrane domain (TM), and the cytosolic domain (CP). Immunodominant regions with either high or low sequence identity with β-coronavirus OC43 are shown. (Panel B and Panel C) The homotrimeric SARS-CoV-2 spike protein is shown in the (B) closed and (C) open state (PDB: 6VXX and 6VYB, respectively). In each protomer of the spike, the protein mainchain is illustrated in white, except for the RBD, which is red. The atoms in the 6 peptide epitopes that were tested are shown as space-filling models, colored according to peptide number. There are regions of missing density in the models, presumably due to conformational flexibility, and these regions are omitted here; CTD1 and S2′FP (are fully resolved; CTD2, S1/S2, and 5′fHR2 are partially resolved and are located as indicated; and HR2 is completely absent in the structure. The sequences in FIG. 4 panel A re as follows: CoV-2 NKKFL-PFQQFGRDIADTTDAV 550-570 (SEQ ID NO:1); OC43 NATYYNSWQNLLYDSNGNLYGF 550-570 (SEQ ID NO:2); CoV-2 604-625 TEVPVAIHADQLTPTWRVYST (SEQ ID NO:3); OC43 NYVFNNSLTRQLQPI (SEQ ID NO:4); CoV-2 660-675 ASYQTQTNSPRRARS (SEQ ID NO:5); OC43 660-675 VDYSKN----RRSRG (SEQ ID NO:6); CoV-2 810-830 ILP----DPSKPSKRSFIEDLLF (SEQ ID NO:7); OC43 810-830 VLGCLGSECSKASSRSAIEDLLF (SEQ ID NO:8); CoV-2 1146-1166 PELDSFKEELDKYFKNHTSPDVDLGDI (SEQ ID NO:9); OC43 PNLPDFKEELDQWFKNQTSVAPDL-SL (SEQ ID NO:10); CoV-2 1182-1209 IQKEIDRLNEVAKNLNESLIDLQELGKYEQYIKWP (SEQ ID NO:11); OC43 LQVEMNRLQEAIKVLNQSYINLKDIGTYEYYVKWP (SEQ ID NO:12).

FIG. 5 panels show SARS-CoV-2 convalescent IgG from mild and severe donors differentially target seasonal CoV-conserved and non-conserved SARS-CoV-2 spike protein immunodominant epitopes. FIG. 5 panels A, B, C, D, E, F, and G show graphs comparing the levels of IgG-binding to (A) RBD, (B) S2′FP, (C) HR2, (D) 5′fHR2, (E) CTD1, (F) CTD2, and (G) S1/S2 regions in CoV-2+ convalescent donors as detected by luminescent ELISA versus severity scores (left) or total CBA anti-spike IgG levels (right). n=28; the SEMs of N=3 experiments are graphed.

FIG. 6 panels show multivariable analysis identifies distinct humoral immune profiles in mild versus severe COVID-19. FIG. 6, panel (A) Scatter matrix chart summarizes the Spearman's correlation (upper) and the scatterplots (lower) between all analyzed variables using the entire cohort (n=28). The Spearman's r values are shown inside the upper squares, and the scale of light-to-dark indicates the strength of the absolute correlation (weak-to-strong). The correlation is indicated as negative or positive by the Spearman r value. The small bar graphs (diagonal) represent the distribution of data for each variable. FIG. 6, panel (B) Biplot shows the principal component analysis (PCA) depicting the mild-scored (n=13) and more severe-scored (n=15) COVID-19 patients, according to their severity scores. FIG. 6, panel (C) The contribution of each variable to PCA for dimension 1 and 2 is represented by bars, and its threshold is indicated as a dotted line. FIG. 6, panels (D and E) Polar plots show the different profiles of humoral response for mild and more severe groups. Each bar in the plot represents the mean of Z scores for each variable.

FIG. 7 panels show the ratio of IgG targeting the HR2/S2′FP regions of the SARS-CoV-2 spike protein correlated with COVID-19 severity in convalescent and ICU-hospitalized patients. FIG. 7 (panel A) anti-SARS-CoV-2 or (panel B) anti-OC43 spike IgG titers as detected by CBA in SARS-CoV-2-naive (CoV-2⁻), non-hospitalized convalescent (CoV-2⁺), and ICU-hospitalized COVID-19 patients (n=20 naive, 28 convalescent, and 17 ICU). FIG. 7 panel C. Comparison of SARS-CoV-2 versus OC43 anti-spike IgG titers in ICU-hospitalized COVID-19 patients. FIG. 7 panels D, E, and F show the ratio of (panel D) HR2/S2′FP, (panel E) RBD/S2′FP, and (panel F) 5′fHR2/S2′FP in convalescent non-hospitalized donors. FIG. 7 panels G and H show the levels of IgG binding to (panel G) HR2 and (panel H) S2′FP regions as detected by luminescent ELISA in SARS-CoV-2-naive (CoV-2⁻), convalescent non-hospitalized donors split by mild (<45), more severe (>45), and ICU-hospitalized COVID-19 patients. FIG. 7 panel I shows the ratio of HR2/S2′FP IgG-binding levels in SARS-CoV-2-convalescent donors versus ICU-hospitalized COVID-19 patients. The SEMs of N=3 experiments are shown.

FIG. 8 panels show comparison of anti-spike IgG levels in convalescent donors as quantified by ELISA vs Cell-based assays. Related to panels of FIG. 1. (Panel A) anti-spike IgG titers in SARS-CoV-2 naïve (CoV-2−) and SARS-CoV-2 positive convalescent (CoV-2+) donors (n=20 and 28, respectively), as quantified by recombinant spike protein ELISA vs Cell-based (CB) binding assay. Horizontal line represents 3-fold above the mean anti-spike levels of naïve (CoV-2−) donors, as quantified by ELISA. Levels of anti-spike IgG titers as quantified by (panel B) ELISA, or (panel C) Cell based IgG binding assay. SARS-CoV-2 naïve (CoV-2-) and convalescent (CoV-2+) donors are shown with convalescent donors split by COVID-19 severity scores. (n=20 naïve, 13 mild, 15 more severe, and 17 ICU). The SEM of N=3 experiments are shown

FIG. 9 panels show levels of anti-spike IgG-induced FcγR-activation correlates with COVID-19 severity and anti-spike titers. Related to FIG. 2. Figure depicts the levels of FcγR-signaling induce by purified IgG derived from SARS-CoV-2 convalescent donors in response to SARS-CoV-2 spike protein expressed on the surface of 293T cells (n=28). Graphs show the levels of (panel A) FcγRIIa and (panel B) FcγRIIIa signaling induced by 25 μg/ml of purified IgG from all SARS-CoV-2 convalescent donors. Horizontal line represents 2-fold above the mean anti-spike levels of all naïve (CoV-2−) donors in each FcγR signaling assay. Scatter plots show the area under the (panel C) FcγRIIa or (panel D) FcγRIIIa signaling curve versus COVID-19 severity scores. Scatter plots show the area under the (panel E) FcγRIIa or (panel F) FcγRIIIa signaling curve versus the levels of anti-spike IgG titers as quantified by cell-based IgG binding assay. Scatter plots show the area under the (panel G) FcγRIIa or (panel H) FcγRIIIa signaling curve versus the levels of anti-spike IgG titers as quantified by ELISA. All FcγR signaling assays were conducted using a three-point titration curve of purified donor IgG (25 ug/ml, 5 ug/ml, & 1 ug/ml). Area under all points was used to calculate AUC. The SEM of N=3 experiments are shown, along with significance of slopes and r² values.

FIG. 10 panels show SARS-CoV-2 convalescent IgG differentially target seasonal CoV-conserved and non-conserved SARS-CoV-2 immunodominant epitopes. Related to FIG. 5. Graphs compare the levels of IgG-binding to (panel A) RBD, (panel B) CTD1, (panel C) S2′FP, (panel D) CTD2 (panel E) HR2, (F) S1/S2 (G) 5′F HR2 regions in SARS-CoV-2 naïve (CoV-2−, n=20) and SARS-CoV-2 positive convalescent (CoV-2+, n=28) donors. Convalescent donors were split into two groups based on COVID-19 severity scores, mild (<45, n=13), and more severe (>45, n=15). The SEM of N=3 experiments are shown.

FIG. 11 panels show preferential antibody-binding to different regions of the SARS CoV-2 spike correlates with COVID-19 severity. Related to FIG. 6. (panel A and panel C) Scatter matrix chart summarizes the Spearman's correlation (r values, upper) and the scatter plots (lower) between all analyzed variables for samples separated by mild (n=13) or more severe (n=15) symptoms, respectively. The small bar graphs (diagonal) represent the distribution of data for each variable. (panel B and panel D) Heatmaps show the Spearman's correlations (r values) between IgG-Spike or IgG-RBD and the levels of IgG targeting the six functional spike domains for samples separated by (panel B) mild (n=13) or (panel D) more severe (n=15) symptoms.

FIG. 12 panels show the ratio of IgG targeting of SARS-CoV-2 spike protein functional domains correlates with COVID-19 disease severity. Specifically, the graphs depict the ratio of IgG-binding to (Panel A) HR2 (pep3)/S2′FP (pep5), (Panel B) RBD/S2′FP (pep5), (Panel C) 5′FHR2 (pep6)/S2′FP (pep5) regions as detected by luminescent ELISA versus disease severity scores. Graphs show the SEM levels of IgG targeting from N=3 experiments. Significance of slopes and r² values are shown.

FIG. 13 panels show the additive ratio of the levels of IgG targeting peptides 3+peptide 6 over the levels of peptide 5 targeting is more significantly correlated with COVID-19 disease severity, as compared to the ratio of peptide 3/5 alone vs disease severity. Specifically, the graphs depict the additive level of IgG binding to HR2 (pep3)+RBD versus COVID-19 severity (Panel A and Panel B). Graphs show the ratio of IgG binding to HR2 (pep3)+RBD/S2′FP(pep5) versus COVID severity scores. All IgG binding levels were the SEM levels of IgG targeting from N=3 experiments as assessed by luminescent ELISA. Significance of slopes and r² values are shown. (Panel B).

FIG. 14 panels show the additive ratio of the levels of IgG targeting peptides 3+peptide 6 over the levels of peptide 5 targeting is more significantly correlated with COVID-19 disease severity, as compared to the ratio of peptide 3/5 or 6/5 alone versus disease severity. Panel A—the additive level of IgG binding to HR2 (pep3)+5′FHR2 (pep6) versus COVID-19 severity. Panel B—graph showing the ratio of IgG binding to HR2 (pep3)+5′FHR2 (pep6)/S2′FP (pep5) versus COVID severity scores. All IgG binding levels were the SEM levels of IgG targeting from N=3 experiments as assessed by luminescent ELISA. Significance of slopes and r² values are shown.

FIG. 15 provides an overview of the assay components and representative and non-limiting examples of uses of the described compositions and methods for use in clinical prognosis and as a companion diagnostic, and medical interventions based on the depicted outcomes.

FIG. 16 provides Table S1, showing characteristics of SARS-CoV-2 convalescent and naïve donors.

FIG. 17 provides Table S2, and shows symptomology of SARS-CoV-2 convalescent donors. Specifically, Table S2 depicts frequency of COVID-19 symptoms experienced by convalescent donors. Symptom intensity was scored out of 10, with 10 being the most severe, and 0 not being experienced. Mild convalescent donors were defined as donors with composite symptom intensity scores below 45. Severe convalescent donors were defined as donors with composite symptom intensity scores above 45.

FIG. 18 provides Table S3. Table S3 shows sequence identity of SARS CoV-2 Immunodominant epitopes and functional regions in comparison to seasonal hCoVs. Related to FIG. 2. The level of sequence identity between spike proteins were assessed using PRALINE software (IBIVU), by comparing SARS-CoV-2 spike protein sequence (Genbank YP_009724390.1) to spike protein sequences of SARS1 (Genbank AAP13567.1), OC43 (Genbank AVR40344.1), HKU1 (YP_173238.1), NL63 (APF29071.1), or 229E (APT69883.1). Percent sequence identity was measured by the level of exact amino acid conservation in reference to the SARS-CoV-2 spike sequence. Gaps in hCoV sequences were treated as no conservation. The sequences in FIG. 18 as provided in Table S3 are as follows:

S1-319-541 -
(SEQ ID NO: 13)
RVQPTESIVRFPNITNLCPFGEVFNATRFASVYAWNRKRISNCVADYSVL
YNSASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFERDI
STEIYQAGSTPCNGVEGFNCYFPLQSYGFQPTNGVGYQPYRVVVLSFELL
HAPATVCGPKKSTNLVKNKCVNF;
S1-556-576 (Pep4) -
(SEQ ID NO: 14)
NKKFLPFQQFGRDIADTTDAV;
S1-618-638; (Pep1) -
(SEQ ID NO: 15)
TEVPVAIHADQLTPTWRVYST;
S1/S2-672-687 (Pep2) -
(SEQ ID NO: 16)
ASYQTQTNSPRRARSV;
S2-805-823 (Pep5) -
(SEQ ID NO: 17)
ILPDPSKPSKRSFIEDLLF;
S2-1143-1167 (Pep6) -
(SEQ ID NO: 18)
PELDSFKEELDKYFKNHTSPDVDLG;
S2-1179-1213 (Pep3)
(SEQ ID NO: 19)
IQKEIDRLNEVAKNLNESLIDLQELGKYEQYIKWP.

### DETAILED DESCRIPTION

Unless defined otherwise herein, all technical and scientific terms used herein have the same meaning as commonly understood by one of ordinary skill in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification includes every narrower numerical range that falls within such broader numerical range, as if such narrower numerical ranges were all expressly written herein.

The singular forms “a”, “an” and “the” include plural referents unless the context clearly dictates otherwise. The terms “a” (or “an”), as well as the terms “one or more,” and “at least one” can be used interchangeably herein. Furthermore, “and/or” where used herein is to be taken as specific disclosure of each of the two specified features or components with or without the other.

All amino acid sequences of the proteins and peptides described herein include sequences having 90.0-99.9% identity across their entire lengths to the described amino acid sequences. The amino acid or polynucleotide sequence as the case may be associated with each GenBank or other database accession number of this disclosure is incorporated herein by reference as presented in the database on the effective filing date of this application or patent.

Aspects of this disclosure include each peptide described herein, and all combinations of such peptides. All amino acid sequences in this disclosure include all segments thereof that are at least 5 contiguous amino acids of the described sequences. The peptides are used in assays as further described herein to provide profiles of antibodies that can bind with specificity to the described peptides. The assays and antibody profiles are used in certain embodiments for predicting an individual's response to SARS-CoV-2 infection, SARS-CoV-2 vaccination, and/or for determining interventions suitable for prophylaxis and/or therapy for SARS-CoV-2 infection. Thus, in certain aspects, the disclosure provides for administering one or more agents to an individual to prevent, inhibit development of, or treat a SARS-CoV-2 infection, based on the determined antibody profiles, as further described herein.

In more detail, the present disclosure reveals antibody signatures that comprise clusters of humoral immune responses with clinical relevance. In embodiments, the described antibody signatures correspond with a decreased risk of severe disease, or an increased risk of severe disease. “Severe disease” is considered to be a SARS-CoV-2 infection that causes significant disease or is lethal, or indicates a need for an intervention, such as admittance to a hospital, ventilation, supplemental oxygen, administration of anti-viral, anti-inflammatory, or other therapeutic agents to treat a SARS-CoV-2 infection, or a combination thereof. Patients experiencing severe disease can present with significant respiratory distress, damage, or failure, including Acute respiratory distress syndrome (ARDS); thrombotic and/or cardiovascular complications; sepsis; and/or other organ dysfunction; decreased oxygen saturation, abnormal cytokines, metabolic abnormalities, increased C-Reactive Protein (CRP) and/or d-dimer levels; etc. Accordingly, the present disclosure demonstrates that ratios of protective to inefficient or otherwise deleterious antibodies (and vice versa) can be used to predict immune responses to SARS-CoV-2 exposure, infection, and vaccination. The described antibody ratios are not necessarily mutually exclusive in any particular individual. In embodiments, it is considered that an inefficient or otherwise deleterious immune response will lead to severe disease, or less than optimal vaccine response. In embodiments, a preexisting (e.g., before SARS-CoV-2 exposure) inefficient immune response is considered to be due inefficient or otherwise deleterious preexisting antibodies developed in response to exposure to one or more different coronaviruses that are not SARS-CoV-2. In contrast, a preexisting efficient immune response, which may prevent the onset of severe diseases, is considered to be due to efficient antibodies developed in response to exposure to one or more other coronaviruses that are not SARS-CoV-2. Upon exposure to SARS-CoV-2, a de novo immune response is considered to also be possible, and is considered to be characterized by higher levels of antibodies targeting regions unique to SARS-CoV-2 (for example, the RBD).

Without intending to be bound by any particular theory, it is considered that antibodies directed to certain combinations of peptides as further described herein indicate a protective SARS-CoV-2 humoral immune recall response, or a protective SARS-CoV-2 de novo humoral response. Likewise, and again without intending to be constrained by any particular interpretation, it is considered that certain antibody ratios as described herein represent an inefficient or otherwise deleterious SARS-CoV-2 humoral immune response.

In general, the disclosure involves testing a biological sample from an individual to determine antibodies which may be produced by exposure to non-SARS-CoV-2 coronaviruses, such as seasonal coronaviruses. The compositions and methods involve the use of certain peptides and combinations of peptides as further described below. Antibodies, if present in the biological sample, bind with specificity to peptides that are present in an assay that is designed to determine the presence, absence, and/or relative amounts (e.g., a ratio) of the antibodies. Thus, in embodiments, the disclosure provides one or a plurality of peptides, wherein a plurality of peptides may be considered a peptide array. Alternatively, the disclosure provides a plurality of substrates, wherein each substrate in the plurality is associated with only one of the described peptides.

Peptides of the disclosure are described by reference to Table A, and elsewhere in this disclosure. For each peptide, which are segments of SARS-CoV-2 spike protein as indicated, an unmodified sequence is shown above a modified sequence. The modifications are for illustration only, as alternative modifications can be made. The modified peptides of Table A are the sequences of the peptides used in the Examples of this disclosure. The bold, non-italicized amino acid are GS linkers. In the RBD, the non-bold, italicized amino acids comprise a representative streptavidin tag that can be used to attach the peptide to biotin. The bold, italicized amino acids comprise a Protein C tag that can be used to detect the peptide if desired using commercially available anti-Protein C antibodies. The poly-histidine tag is at the C-terminus and is not annotated. Peptides 1, 2, 3, 4, 5 and 6 are also referred to herein as Pep1, etc., and pep1, etc. The receptor binding domain peptide is referred to as RBD.

TABLE A

| | |
|---|---|
| Receptor | TNLCPFGEVFNATRFASVYAWNRKRISNCVADYSVLYNSASFSTF |
| Binding | KCYGVSPTKLNDLCFTNVYADSFVIRGDEVRQIAPGQTGKIADYN |
| Domains (RBD) | YKLPDDFTGCVIAWNSNNLDSKVGGNYNYLYRLFRKSNLKPFER |
| | DISTEIYQAGSTPCNGVEGFNCYFPLQSYGFQPTNGVGYQPYRVV |
| | VLSFELLHAPATVCGPK (SEQ ID NO: 20); |
| | TNLCPFGEVFNATRFASVYAWNRKRISNCVADYSVLYNSASFSTF |
| | KCYGVSPTKLNDLCFTNVYADSFVIRGDEVRQIAPGQTGKIADYN |
| | YKLPDDFTGCVIAWNSNNLDSKVGGNYNYLYRLFRKSNLKPFER |
| | DISTEIYQAGSTPCNGVEGFNCYFPLQSYGFQPTNGVGYQPYRVV |
| | VLSFELLHAPATVCGPKGSGGLNDIFEAQKIEWHEGSGEDQVDPR |
| | LIDGKGSGHHHHHHHHHH (SEQ ID NO: 21) |
| Peptide 1 | TEVPVAIHADQLTPTWRVYST (SEQ ID NO: 22); |
| C-terminus | GSGSTEVPVAIHADQLTPTWRVYSTGSGSGS (SEQ ID NO: 23) |
| domain (CTD2) | |
| Peptide 2 | ASYQTQTNSPRRARSV (SEQ ID NO: 24); |
| Furin cleavage | GSGSASYQTQTNSPRRARSV (SEQ ID NO: 25) |
| site (S1/S2) | |
| Peptide 3 | IQKEIDRLNEVAKNLNESLIDLQELGKYEQYIKWP (SEQ ID |
| Heptad Repeat- | NO: 26); GSGSIQKEIDRLNEVAKNLNESLIDLQELGKYEQYIKWP |
| 2 (HR2) region | (SEQ ID NO: 27) |
| Peptide 4 | NKKFLPFQQFGRDIADTTDAV (SEQ ID NO: 28); |
| C-terminus | GSGSNKKFLPFQQFGRDIADTTDA (SEQ ID NO: 29) |
| domain (CTD1) | |
| Peptide 5 | ILPDPSKPSKRSFIEDLLF (SEQ ID NO: 30); |
| S2′ Fusion | GSGSILPDPSKPSKRSFIEDLLF (SEQ ID NO: 31) |
| protein (S2′FP) | |
| Peptide 6 | PELDSFKEELDKYFKNHTSPDVDLG (SEQ ID NO: 32); |
| 5′ HR2 flanking | GSGSPELDSFKEELDKYFKNHTSPDVDLG (SEQ ID NO: 33) |
| region (5′fHR2) | |

In embodiments, a plurality of substrates of the disclosure comprises or consists of at least two substrates, said substrates having bound thereto one of the RBD, peptide 1 (pep1), peptide 2 (pep2), peptide 3 (pep3), peptide 4 (pep4), peptide 5 (pep5), and peptide 6 (pep6). In alternative embodiments, additional peptides or proteins may be included. In embodiments, the disclosure comprises exposing a biological sample to a plurality of substrates. In embodiments, the substrates comprises a combination of 2-7 substrates, each with a different and optionally only one peptide associated with each substrate. Thus, each substrate in the plurality of substrates may comprise or consist of 2, 3, 4, 5, 6, or 7 substrates. In embodiments, the described peptide combinations may be the only peptides present in the assay.

In embodiments, plurality of substrates have a peptide component that is at least RBD, pep3, pep5, pep6, or any combination thereof. In non-limiting embodiments, plurality of substrates comprises or consists of a peptide component that is a combination of pep3 and pep5, a combination of RBD and pep5, a combination of pep5 and pep6, a combination of pep3 and RBD, a combination of pep3, RBD and pep5, a combination of pep3 and pep6, or a combination of pep3, pep6 and pep5. In embodiments, any of the described peptide combinations are used to define antibody profiles that predict severity of a SARS-CoV-2 infection and/or the efficacy of vaccination. In one embodiment, antibody titers against specific peptides are compared. In this regard, as can be seen from the accompanying figures, in embodiments, ratios of antibodies to certain peptide combinations can be used to predict severity. The severity scores in these figures having been calculated as described below in Example 1. In embodiments, the following peptide combinations can be used to determine antibodies that bind to the peptides to produce, if desired, a predicted severity risk and, depending on the outcome, provide an intervention, examples of which are described below: 1) A ratio of pep3 antibodies to pep5 antibodies. 2) A ratio of RBD antibodies to pep5 antibodies. 3) A ratio of pep6 antibodies to pep5 antibodies. 4) A ratio of pep3 antibodies to RBD antibodies, 5) A ratio of a combination of pep3 and RBD antibodies to pep5 antibodies. 6) A ratio of pep3 antibodies to pep6 antibodies. 7) A ratio of pep3 and pep6 antibodies to pep5 antibodies. Thus, the disclosure provides for predicting severity risk.

In one aspect, determined levels of antibodies to RBD are inversely correlated with disease severity. In another aspect, a greater proportion of antibodies to pep3 are considered to represent a protective memory response. In another aspect, pep5 dominant Ab profiles indicate a deleterious recall response. In another aspect, a ratio of pep3 and RBD antibodies to pep5 antibodies provides a more accurate prediction of severity than that achieved using a ratio of pep3 antibodies to pep5 or RBD antibodies to pep5. In another embodiment, a ratio of pep3 and pep6 antibodies to pep5 antibodies provides a more accurate prediction of severity than that achieved using a ratio of pep3 to pep5 antibodies or pep6 to pep5 antibodies.

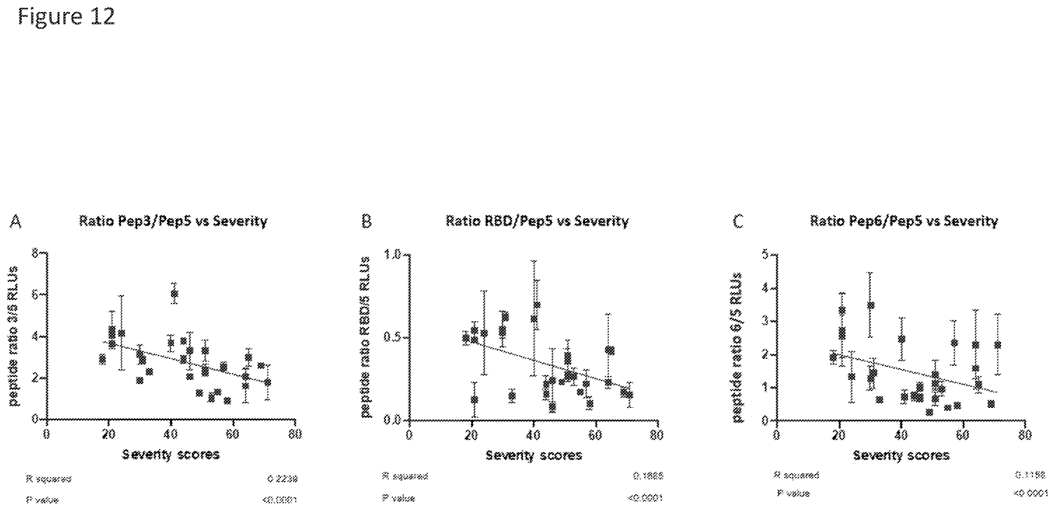

Representative data showing correlations of severity for antibody ratios for pep3 to pep5 severity are summarized in FIG. 12, panel A. Representative data showing correlations of severity for antibody ratios for RBD to pep5 are shown in FIG. 12, panel B. Representative data showing correlations of severity for antibody ratios for pep6 to pep5 are shown in Figure A, panel C. In an embodiment, the disclosure provides method of calculating a ratio of conserved-protective Abs (and/or de novo Abs) relative to conserved-inefficient Abs to predict disease severity and/or vaccine efficacy.

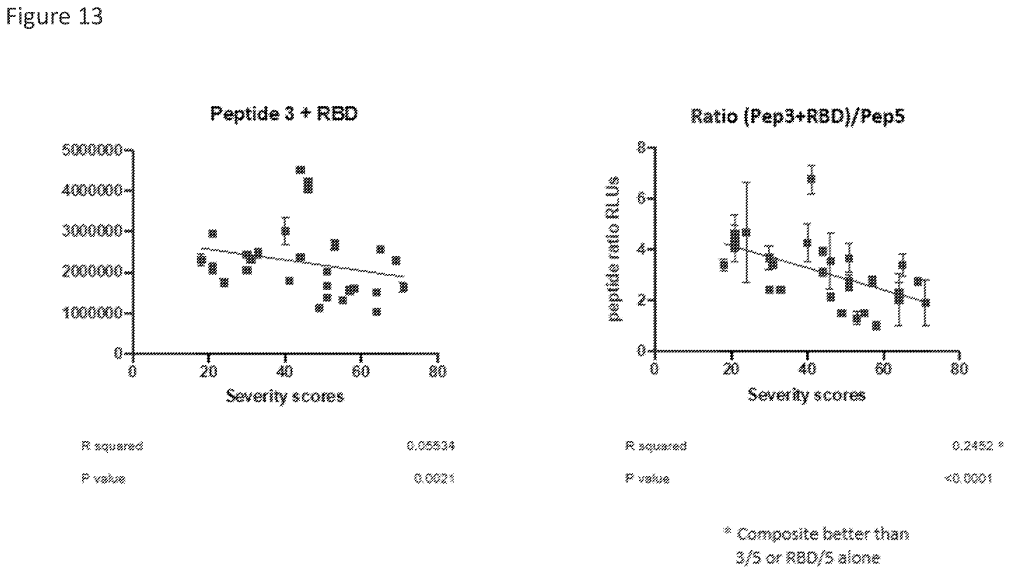

Representative data showing correlations of severity for antibody ratios for pep3 and RBD are shown in FIG. 13, left panel. Representative data showing correlations of severity for antibody ratios for the combination of pep3 and RBD relative to pep5 are shown in FIG. 13, right panel.

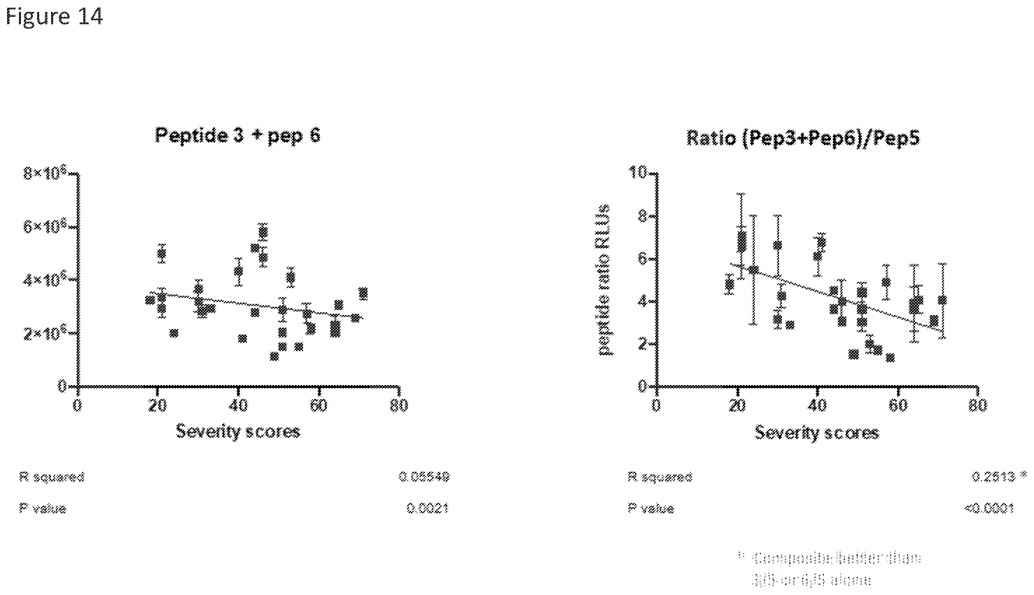

Representative data showing correlations of severity for antibody ratios for antibodies to pep3 and pep6 are shown in FIG. 14, left panel. Representative data showing correlations of severity for antibody ratios for the combination of pep3 and pep6 to pep5 are shown in FIG. 14, right panel.

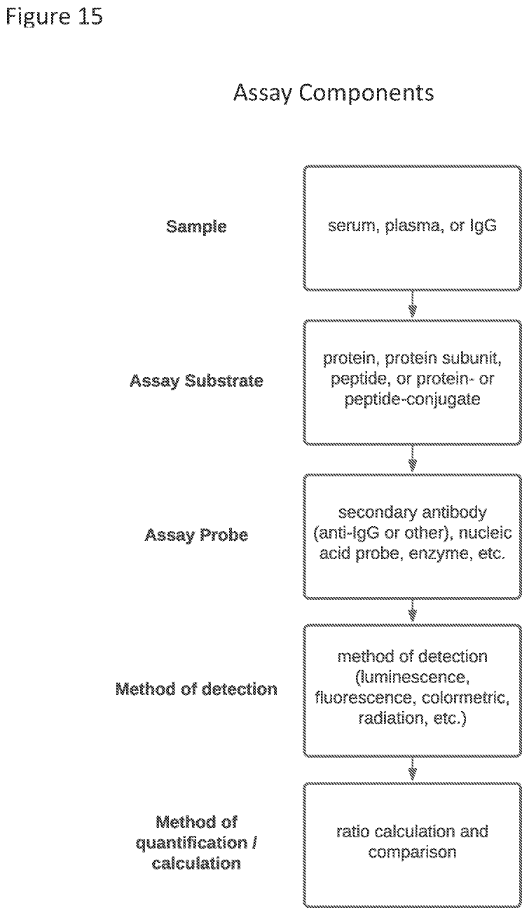

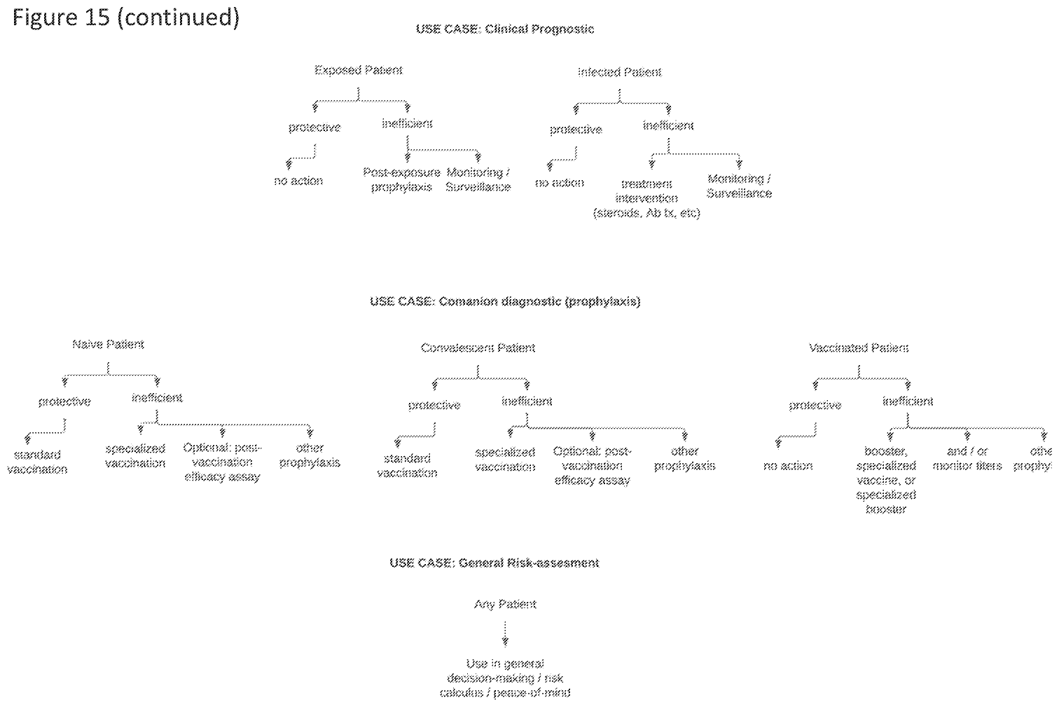

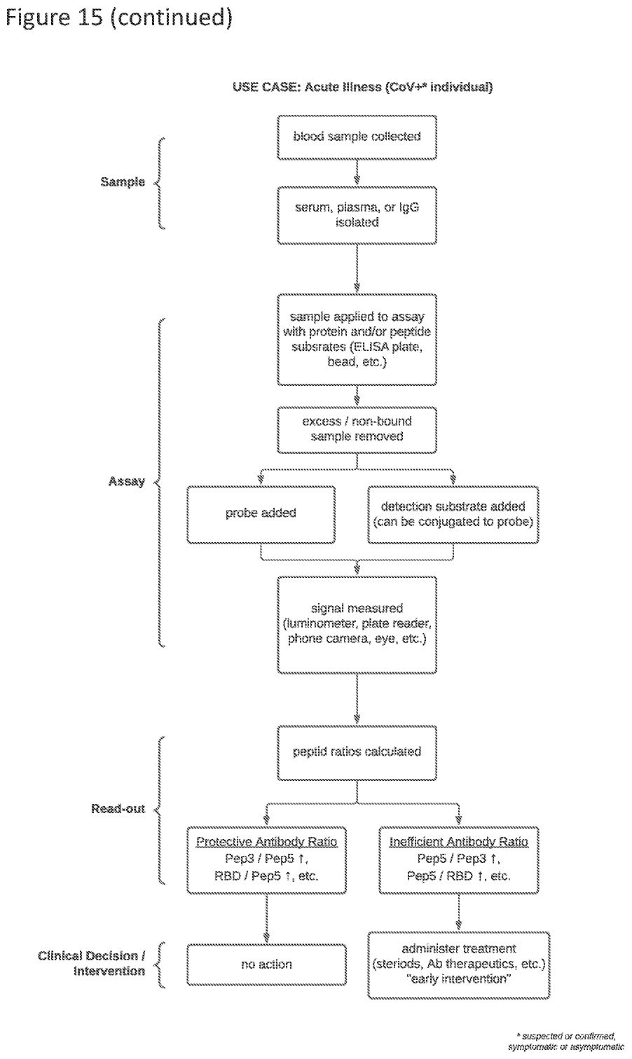

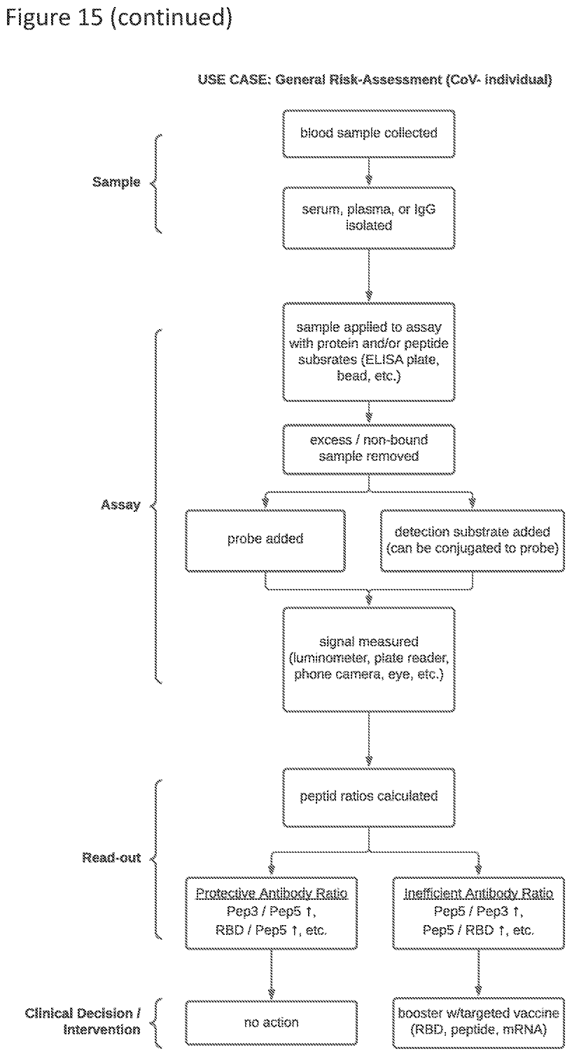

FIG. 15 provides an overview of the assay components and representative and non-limiting examples of uses of the described compositions and methods for use in clinical prognosis and as a companion diagnostic.

In embodiments, antibodies directed to the described peptide combinations as provided on separate substrates may be analyzed as a fraction of total Ig in a sample, which may include total IgG. The total IgG may include any IgG subclasses, e.g. IgG1, IgG2, IgG3, IgG4, and combinations thereof. In certain non-limiting embodiments, the sample may also contain any class of antibodies that are IgA, IgD, IgE or IgM antibodies, and all subclasses thereof.

In embodiments, the described peptides can be modified using any suitable approach, including but not limited to including one or more purification tags, including but not limited to a His-tag. In an embodiment, a His-tag is a linear sequence of n histidine residues where n is typically 6-10. His-tags achieve purification by binding specifically to nickel or cobalt ions, which may be for example, attached to a substrate, such as any suitable beads. The His-tag, or any other suitable purification tag, may be placed at the N-terminus, or at the C-terminus of the peptide. In embodiments, a FLAG-tag, or FLAG octapeptide, or FLAG epitope, may be included. Suitable FLAG sequences are known in the art. In embodiments, a Small ubiquitin-related modifier (SUMO) tag, such as a His-SUMO tag can be included.

In embodiments, one or more of the peptides comprise a linker. The term “linker” refers to a chemical moiety that connects a segment the peptide to another material. Linkers include amino acids, but other linkers are encompassed as well. Generally speaking, amino acid linkers may be principally composed of relatively small, neutral amino acids, such as Glycine, Serine, and Alanine, and can include multiple copies of a sequence enriched in Glycine and Serine. In embodiments, the linker is provided as a means to connect the peptide to a substrate. As such, the linker may be attached to a binding partner that connects the peptide to a substrate. Non-limiting examples of binding partners include streptavidin, biotin,

In embodiments, one or more of the peptides are modified so that they can be attached to a substrate. The attachment may or may not be via a linker. In various embodiments, a peptide can be reversibly or irreversibly attached to a substrate, such as by being covalently, ionically, or physically bound to a, for example, a solid-phase substrate using methods such as covalent bonding via an amide or ester linkage, ionic attraction, or by adsorption, non-limiting examples of which comprise biotin and streptavidin tags. The substrate can be any suitable substrate onto which one or more described peptides can be attached. Examples include substrates typically used in immunodetection assays, lateral flow devices, bead-based assays, microfluidic devices, etc. Thus, a solid substrate can be a porous solid substrate that allows the flow of liquid through the substrate. The liquid can flow through the porous substrate via any suitable means, such as by capillary action, microfluidics, etc. The substrate can also be a non-porous solid substrate, such as beads formed from glass or other non-porous materials, such as plastic. Thus, in embodiments, a plurality of substrates may include a mixture of, for example, beads, that are attached to different peptides. In an embodiment, a single substrate, such as a bead, may have two or more peptides attached to it. In certain embodiments, the substrate, the peptide(s), or both may be functionalized to facilitate peptide attachment. The substrate may be blocked prior to attaching the peptides to reduce artifacts. The describes assays may also be performed in solution.

In embodiments, one or more of the peptides are present in vitro, such as in an in vitro assay. In embodiments, the peptides are in contact with a biological sample obtained from an individual. In embodiments, the biological sample is from an individual who has been exposed to one or more coronaviruses that are not a type of SARS-CoV-2. In embodiments, the individual from whom a first sample was obtained has not been previously (e.g., before obtaining the first biological sample that is assayed) infected with SARS-CoV-2. In embodiments, the individual from whom a first sample was obtained has not been previously vaccinated against SARS-CoV-2. In embodiments, the individually has been previously vaccinated against SARS-CoV-2, and/or has been infected with SARS-CoV-2, or is infected with SARS-CoV-2 at the time the sample is obtained. In embodiments, the individual from whom the biological sample is obtained is at risk of exposure to SARS-CoV-2, or is suspected of having or at risk of developing COVID-19, or has been diagnosed with COVID-19.

The biological sample that is analyzed can be used directly, or subjected to one or more processing steps to render the sample suitable for antibody testing. The sample may be a liquid biological sample, such as a sample of blood or serum, provided the sample comprises antibodies. The antibodies analyzed are not particularly limited. In general, circulating antibodies are determined and generally predominantly comprise IgG isotype antibodies.

Determination of the antibodies that bind to the described peptides can be performed using any suitable approach, preferably any type of enzyme-linked immunosorbent assay (ELISA) assay, including but not limited to a direct ELISA, a sandwich ELISA, a competitive ELISA, and a reverse ELISA. In embodiments, one or more peptides described herein can be incorporated into an immunodiagnostic device, such as a microfluidic device, a lateral flow device, and the like.

In certain embodiments a result obtained from using a method and/or device and/or system of this disclosure can be compared to any suitable reference, examples of which include but are not limited to control sample(s), a standardized curve(s), and/or experimentally designed controls such as a known input value. In embodiments, the patient sample itself comprises a control due to the sample comprising a mixture of antibodies, which may include total IgG in the sample, wherein comparative amounts of the antibodies that bind to distinct peptides are used as a control. Any signal used for determining the antibodies can be normalized to account for, for instance, differing peptide lengths which may comprises a different number of epitopes relative to other peptides that are used in the assay, thereby producing more antibodies bound to a longer peptide relative to a shorter peptide or different overall levels in recall vs de novo antibody responses. Thus, in embodiments the disclosure provides normalizing any result such that the result determines relative proportions of bound antibodies, including but not limited to antibody ratios. In embodiments, analysis of the described antibodies provides for predicting FcγR activation in an individual from whom the antibodies were obtained.

In embodiments, the disclosure provides for a device configured to generate a signal that is based on antibodies bound to the described peptides. In embodiments, the signal is optically accessible. In embodiments, at least some antibodies, if present in the biological sample, are bound to at least some of the peptides in the plurality of proteins, e.g., the peptide array. In embodiments, the antibodies are bound to the proteins, and the assay further comprises detectably labeled antibodies bound to the antibodies that are bound to the peptides, such as in an aforementioned ELISA assay. Thus, in one approach, the disclosure includes determining a signal from the detectably labeled antibodies. The detection antibodies can be labeled using any suitably detectable label. In embodiments, the detectable labels produce a signal that comprises UV light (<380 nm), visible light (380-740 nm) or far red light (>740 nm). In embodiments, the detectable signal comprises a fluorescent signal. In embodiments, relative light units (RLUs) are used to determine ratios of signals from different antibodies, such as RLUs determined from a luminescent ELISA. In embodiments, a cell-based assay described below can be used in the methods of the disclosure.

The signal may be interpreted using any suitable device. In embodiments, any suitable imager located proximal to analyzed sample can be used. In embodiments, free-space optics may be used to detect a signal from the assay using any suitable signal detection device that is placed in proximity to the location where a detectable signal is generated, such as a CCD camera. In embodiments, the disclosure provides a microfluidic device for use in sample analysis. In embodiments, the microfluidic device may comprise, among other features, an optical waveguide to transmit a signal to any suitable measuring device such that optical accessibility to sample is not necessarily required to detect the signal. In embodiments, lens-less optics, and/or a cell phone-based imaging approach is used. In embodiments, one or more segments of an assay device on which the described antibody analysis is performed can be connected to or in communication with a digital processor and/or a computer running software to interpret the presence, absence, amount, or a ratio of antibodies that bind to one or more of the described peptides. The processor may run software and/or implement an algorithm to interpret an optically detectable signal, and generate a machine and/or user readable output. In an embodiment, an assay device used to perform the antibody analysis can be integrated or otherwise inserted into an adapter that comprises a detection device, such as a camera, or a microscope, including but not limited to a fluorescent microscope. In embodiments, a computer readable storage medium can be a component of an assay device of this disclosure, and can be used during or subsequent to performing any assay or one or more steps of any assay described herein. In embodiments the computer storage medium is a non-transitory medium, and thus can exclude signals, carrier waves, and other transitory signals.

In certain embodiments a result based on a determination of the presence, absence, amount, type, ratio, or a combination thereof for antibodies analyzed using an approach and/or a device of this disclosure is obtained and is fixed in a tangible medium of expression, such as a digital file, and/or is saved on a portable memory device, or on a hard drive, or is communicated to a web-based or cloud-based storage system. The determination can be communicated to a health care provider for the purpose of recommending or not recommending any particular medical intervention.

In certain examples the disclosure comprises an article of manufacture, which in embodiments can also be considered kits. The article of manufacture comprises at least one component for use in the antibody analysis described herein, and packaging. The packaging can contain any peptides or combinations thereof described herein, and may further provide reagents for use in determining antibodies, and/or for sample collection. The kit can be provided as a component of a cartridge or similar component, or for example a multi-well plate. Such components may be provided pre-loaded with any combination of reagents required to perform the described analysis. Such components may be provided with the substrate and certain reagents as a lyophilized form for reconstitution in, for example, a suitable immunodiagnostic buffer. In various embodiments, the article of manufacture includes printed material. The printed material can be part of the packaging, or it can be provided on a label, or as paper insert or other written material included with the packaging. The printed material provides information on the contents of the package, and instructs a user how to use the package contents for antibody analysis.

In embodiments, the described peptides may be provided as pharmaceutical formulations. A pharmaceutical formulation can be prepared by mixing the peptides with any suitable pharmaceutical additive, buffer, and the like. Examples of pharmaceutically acceptable carriers, excipients and stabilizers can be found, for example, in Remington: The Science and Practice of Pharmacy (2012) 22nd Edition, Philadelphia, PA. Lippincott Williams & Wilkins, the disclosure of which is incorporated herein by reference. In embodiments, the pharmaceutical formulation comprises a vaccine. In an embodiment, the vaccine comprises a suitable adjuvant, many of which are known in the art.

In certain approaches, a described peptide is modified for prophylactic or therapeutic approaches such that it comprises one or more glycans, and/or by addition of amino acids that comprise Th epitope(s), and/or the peptide is stapled, and/or is cyclicized, and/or is multimerized. Thus, the disclosure provides vaccine formulations for use in prophylaxis and/or treatment for SARS-CoV-2 infection, which may be based at least in part on a determination of preexisting or newly generated antibodies that bind to any of the described peptide combinations.

In embodiments, an effective amount of a one or more peptides described herein, or another suitable agent, is administered to an individual, based at least upon an antibody profile obtained from determining antibodies that bind to the describe peptides. An effective amount means an amount of the described compound(s) that will elicit the biological or medical response by a subject that is being sought by a medical doctor or other clinician. In embodiments, an effective amount means an amount sufficient to prevent, or reduce by at least about 30 percent, or by at least 50 percent, or by at least 90 percent, any sign or symptom of viral infection. In embodiments, fever is prevented or is less severe than if agent(s) had not been administered to an infected individual. In embodiments, viral pneumonia is inhibited or prevented in an infected individual. In embodiments, transmission of the virus from an infected individual to a non-infected individual is inhibited or prevented. In embodiments, the disclosure thus includes administering one or more agents to the individual to treat, prevent development of, or lessen the severity of a SARS-CoV-2 infection. Such agents include but are not necessarily limited to passive immunotherapies such as anti-SARS-CoV-2 antibodies, including but not limited to polyclonal and monoclonal antibodies, single heavy chain only antibodies, diabodies, and vaccination with a protein or peptide described herein, or another type of SARS-CoV-2 vaccine, and anti-viral compounds, such as Paxlovid, Molnupiravir, Remdesvir, and Galidesivir, and other drugs used in the standard treatment of COVID-19, such as steroids, anti-inflammatory drugs, or anticoagulants.

In embodiments, the described assays are used as a clinical diagnostic, or a companion diagnostic so that an intervention can be made based at least in part on the outcome of the assay. In an embodiment, the assay is performed prior to SARS-CoV-2 vaccination or booster administration to analyze, for example, pre-existing coronavirus antibodies to determine what type of vaccine the individual should receive. In one aspect, the vaccine boosts a recall response, and thus may be a vaccine that stimulates production of antibodies that bind to pep3. Such a vaccine may be a single dose vaccine. In another aspect, the vaccine may be designed to supersede pre-existing recall response to pep5, and thus may also comprise a vaccine that stimulates production of antibodies that bind to pep3 or RBD. In embodiments, based on determining an antibody profile as described herein, the individual may be recommended for and administered an initial vaccine, such as a first vaccination, or a booster vaccine, such as a second or third vaccine administration. In embodiments, the described compositions and methods are used to predict a vaccine for use in providing a protective immune response. In embodiments, the vaccine comprises an mRNA vaccine, a protein or peptide vaccine, or a vaccine that is administered using a modified viral vector, including but not limited to live attenuated viral vectors, recombinant viral vectors, and the like. The disclosure also comprises monitoring individuals post-SARS-CoV-2 infection, and post-SARS-CoV-2 vaccination, and using the described compositions and methods to recommend and administer vaccine formulations to improve humoral immune responses, such as vaccines that specifically induce HR2- or RBD-targeting or other efficient antibody responses and/or specifically avoid increasing pre-existing inefficient antibody responses (such as against pep5), or to recommend and administer other anti-viral drugs or biologics to an individual in need thereof.

The following Examples are intended to illustrate but not limit the disclosure.

### EXAMPLE 1

### Non-Hospitalized SARS-CoV-2 Convalescent Individuals Display a Spectrum of COVID-19 Symptom Severity

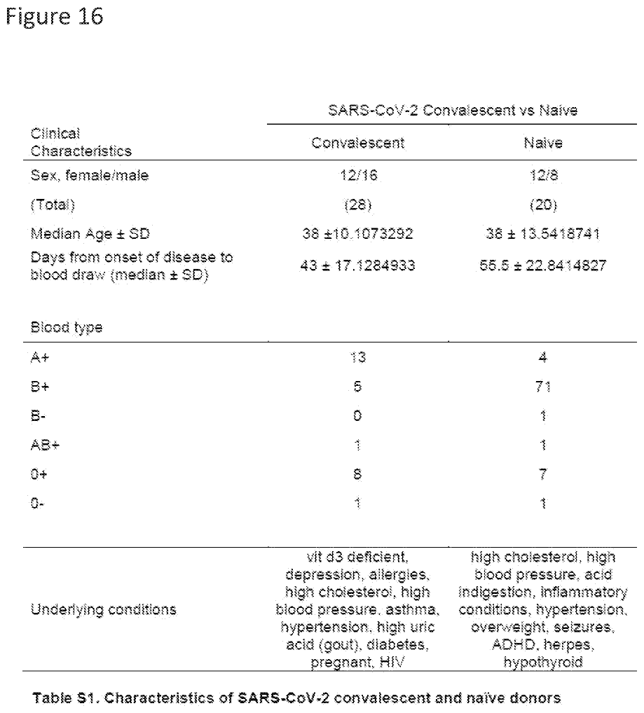

To obtain a better understanding of the humoral responses generated against SARS-CoV-2, a total of 48 blood donors were recruited during the first wave of the COVID-19 pandemic in the spring of 2020. The cohort was separated into two groups based on the history of a positive or negative SARS-CoV-2 test (PCR or serology): convalescent donors (positive test; convalescent) and negative controls (negative test; naive). The negative control group was composed of age- and sex-matched SARS-CoV-2-naive donors (Table S1 as shown in FIG. 16). Importantly, in convalescent donors, the median time from the onset of symptoms to the time of blood draw was 43 days, ensuring that anti-SARS-CoV-2 IgG levels were sufficiently high for pathogen-specific IgG analysis.

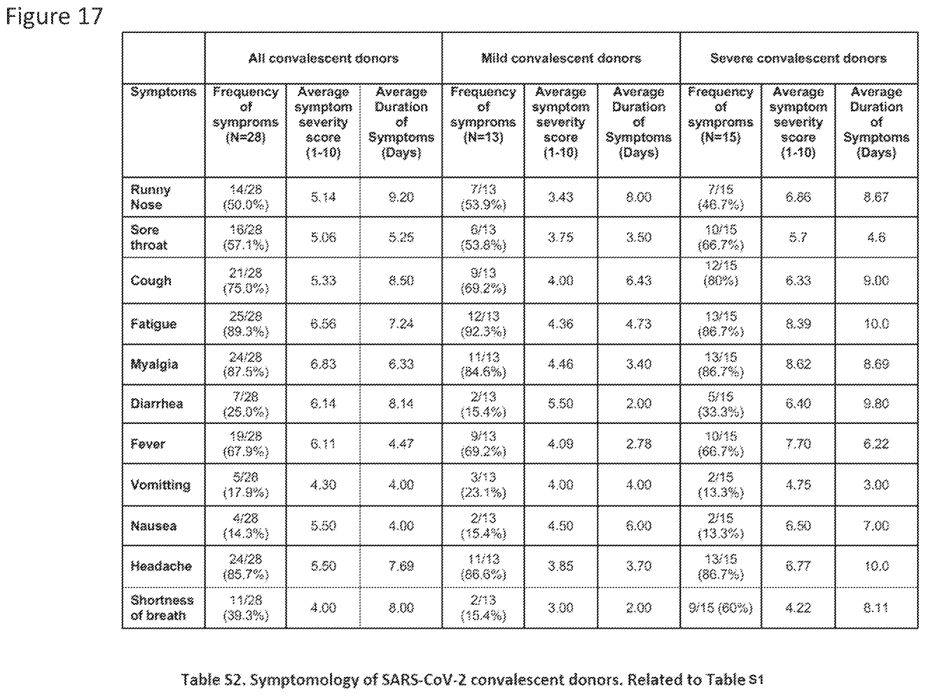

To assess the relative severity of disease, we surveyed the symptom history of each convalescent donor, including the intensity and duration of each symptom. This information was used to calculate both an average severity score for each symptom (Table S2 as shown in FIG. 17) and a composite symptom severity score for every convalescent donor. In doing so, we observed that the convalescent group presented a wide range of symptoms and severity ranging from mild to more severe COVID-19. Moreover, we observed two categories of convalescent donors: those who experienced milder symptoms (n=13), with composite severity scores below 45, and those with more severe disease (n=15), with scores above 45. For simplicity, these two groups are henceforth referred to as mild and severe, respectively, as they reflect these two ends of the non-hospitalized COVID-19 spectrum, with the caveat that all of the donors represent non-hospitalized, non-fatal COVID-19 cases. Despite not being hospitalized, individuals with the highest severity scores (>45) commonly experienced high fevers (>37.8° C.) for more than 1 week, severe myalgia, headaches, and difficulty breathing. In addition, two severe patients experienced weight loss of >15% of body weight.

### EXAMPLE 2

### COVID-19 Severity Correlates With Higher Anti-Spike Ab Titers in SARS-CoV-2 Convalescent Individuals

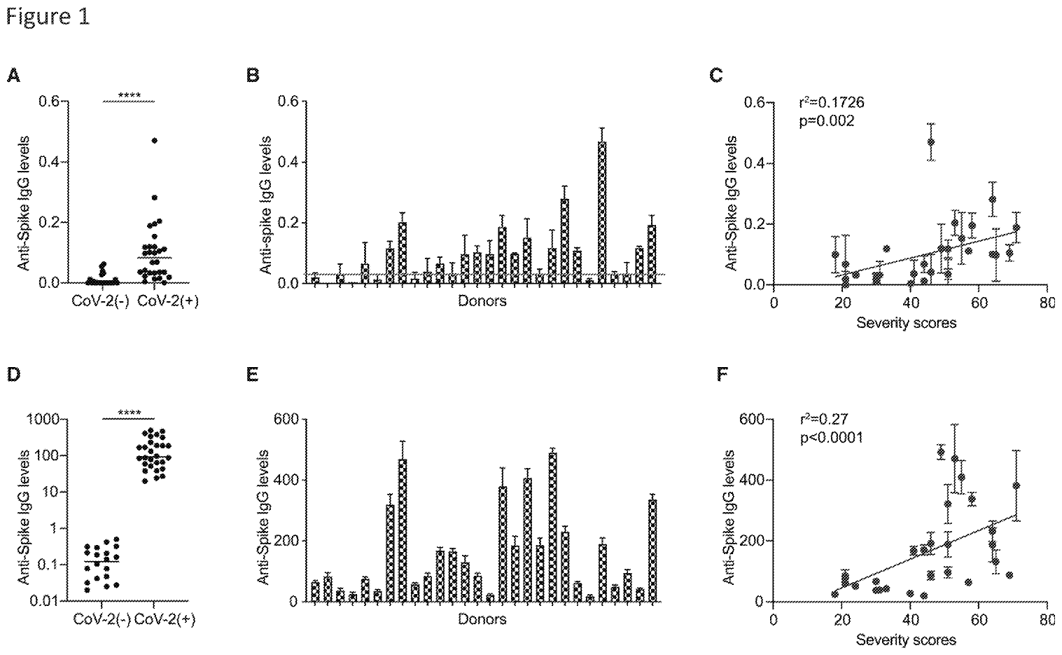

Next, we quantified the levels of IgG directed against the SARS-CoV-2 spike protein in convalescent donors by ELISA (Okba et al., 2020). We detected significantly higher levels of anti-spike IgG in the convalescent versus SARS-CoV-2-naive donors (FIG. 1 panel A). Among the convalescent donors, we observed a range of anti-spike IgG levels, which differed by approximately 30-fold between the lowest and highest samples (FIG. 1 panel B). Interestingly, 2 donors who tested positive for SARS-CoV-2 infection by PCR possessed no detectable anti-spike titers and 8 additional convalescent donors possessed titers that were less than 3-fold above background, which was comparable to the anti-spike levels observed in some SARS-CoV-2-naive donors. In comparing anti-spike IgG levels against convalescent donor severity scores, we observed a significant positive correlation (FIG. 1 panel C). Several studies have found similar correlations between anti-spike IgG and COVID-19 severity in hospitalized patients (Liu et al., 2020; Qu et al., 2020; Young et al., 2020; Zhang et al., 2020).

In addition to neutralization, non-neutralizing effector IgG responses against virus-infected host cells involve the recognition of viral antigens expressed on the surface of host cells (Forthal and Moog, 2009). Therefore, we quantified the levels of anti-spike IgG binding to cell surface-expressed forms of the SARS-CoV-2 spike in 293T endothelial cells transfected to express the SARS-CoV-2 spike. In this system, the levels of anti-spike IgG are quantified using a binding index that accounts for both the percentage and median fluorescence intensity (MFI) of IgG bound to spike-expressing cells. We have previously used this method to discern viral antigen-specific IgG levels in convalescent donors (Alvarez et al., 2014, 2017; Garrido et al., 2018, the disclosures of which are incorporated herein by reference). The benefit of this composite metric is that both the frequency and density of IgG-antigen binding are captured, as both parameters contribute to the activation of non-neutralizing effector functions. In addition, cell surface-expressed spike protein likely retains a tertiary structure and glycosylation that are consistent with natural infection, potentially capturing a greater range of paratopes (i.e., higher sensitivity).

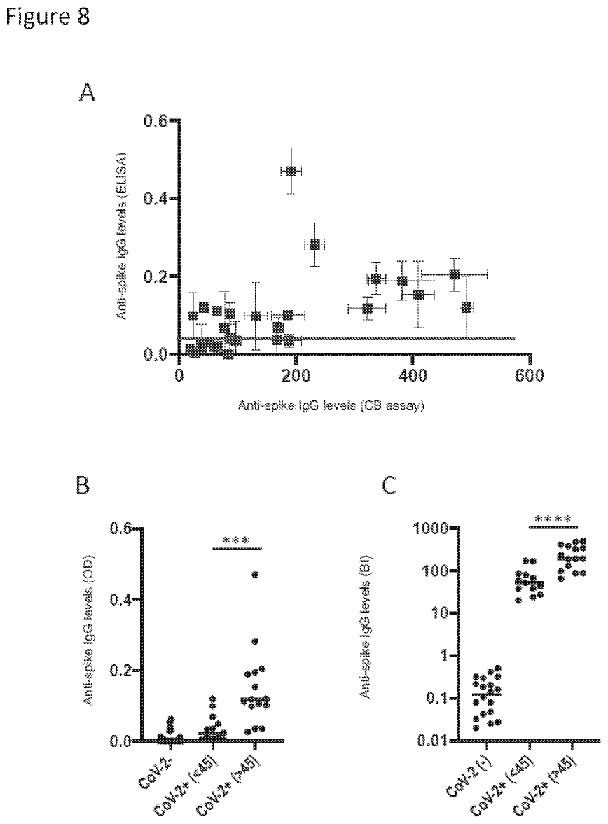

Using this cell-based assay (CBA), we detected an approximately 1.5-log difference in anti-spike IgG titers between naive and SARS-CoV-2 convalescent donors, and a further 2-log difference among the convalescent donors (FIG. 1 panel D and 1 panel E). Interestingly, the two PCR-positive SARS-CoV-2 donors whose anti-spike IgG levels were undetectable by ELISA had detectable, albeit low, anti-spike IgG levels in the cell-based assay. These levels were 1 log above the values obtained with IgG from naive donors, demonstrating greater resolution of the cell-based assay compared to ELISA. In addition, anti-spike IgG levels were readily detectable above negative control (naive) IgG levels in the eight convalescent donors who possessed low-to-negative anti-spike IgG levels using the ELISA assay (FIG. 8 panel A). Other studies have similarly observed higher sensitivity with cell-based detection assays (Fafi-Kremer et al., 2020; Grzelak et al., 2020), which may be attributable to the display of conformational epitopes that are dependent on the tertiary or quaternary structure of the natively folded trimeric spike protein. Altogether, we observed a strong positive correlation between anti-spike IgG levels and disease severity (R2=0.27, p<0.0001) (FIG. 1 panel F), with severe donors possessing significantly higher anti-spike IgG levels (FIG. 8 panel B and panel C).

### EXAMPLE 3

### Higher Levels of Anti-Spike IgG-Mediated FcγR Activation Correlate With COVID-19 Severity

Elevated levels of anti-spike IgG and proinflammatory cytokines are detected in severe hospitalized COVID-19 patients, suggesting that IgG may exacerbate disease severity via FcγR-mediated ADI effects. Therefore, we next examined the levels of Fc-γ receptor IIa and IIIa signaling (FcγRIIa, FcγRIIIa), since both of these FcγRs activate several important cellular effector functions, such as ADCC and ADCP, and are capable of activating an array of proinflammatory cytokines (Nimmerjahn and Ravetch, 2010; Vogelpoel et al., 2015).

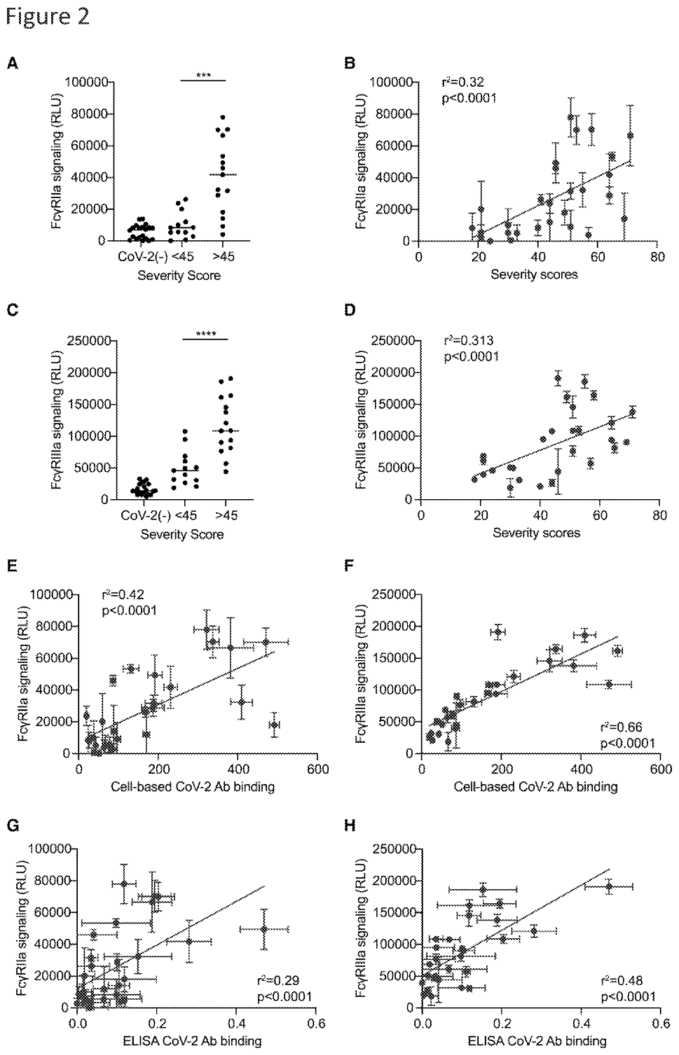

To quantify FcγR activation, we transfected 293T cells to express the SARS-CoV-2 spike and co-cultured these cells with FcγRIIa or FcγRIIIa reporter cell lines in the presence of various concentrations of purified donor IgG (Alvarez et al., 2014, 2017). Using this system, we observed a strong positive correlation between severity scores and both FcγRIIa and FcγRIIIa activation across convalescent donors (FIG. 2 panels BD, 9 panel C, and 9 panel D), with higher overall FcγR signaling in severe versus mild donors (FIG. 2 panel A and panel C). Only background levels of FcγR activation were detected in response to SARS-CoV-2-naive donor IgG (FIG. 2A). Interestingly, the majority of IgG from mild donors did not induce greater FcγRIIa activation than SARS-CoV-2 naive IgG (FIG. 2 panel A). In contrast, both mild and severe convalescent donors induced FcγRIIIa signaling levels that were at least 2-fold above naive control IgG (FIG. 2 panel C).

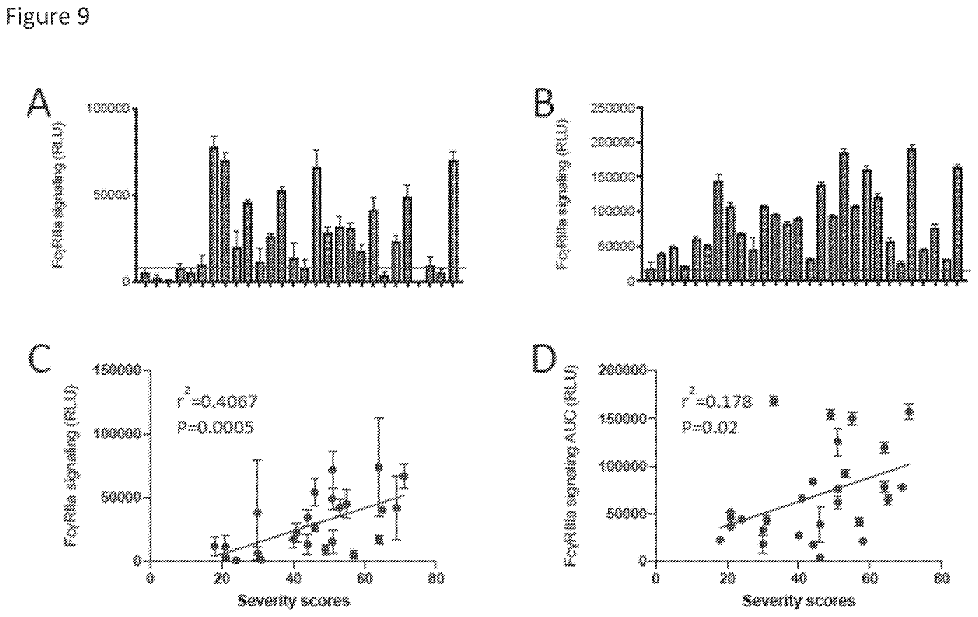

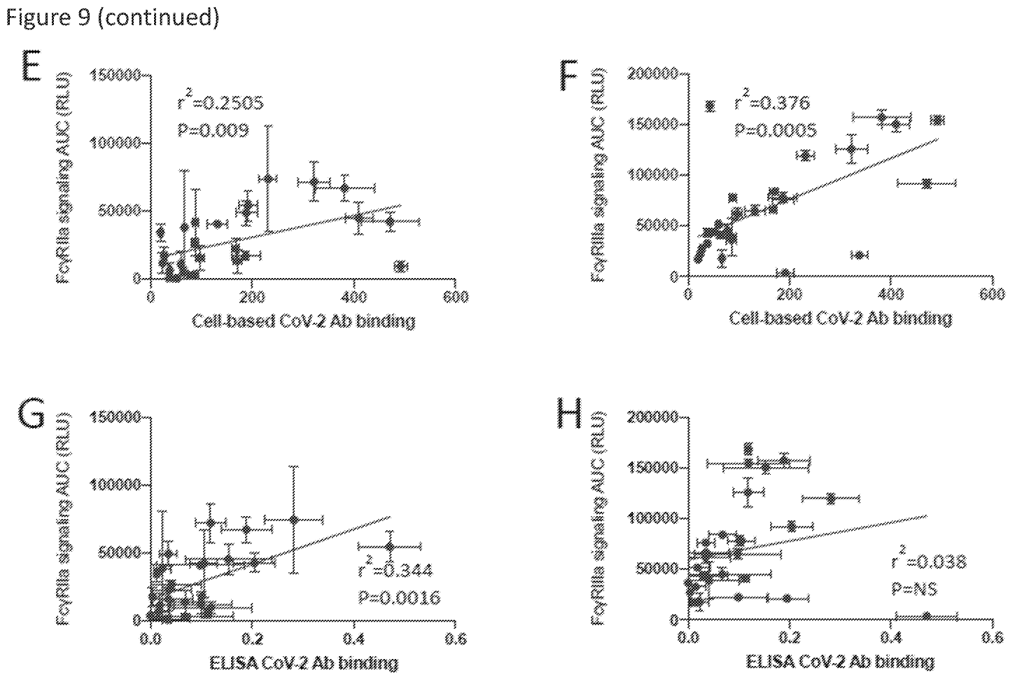

Next, we evaluated the relationship between FcγR activation and the levels of anti-spike IgG. We observed a significant positive correlation with both FcγRIIa and FcγRIIIa signaling and the levels of anti-spike IgG in all convalescent donors, using both ELISA and CBA anti-spike IgG-binding assays (FIG. 2 panels E-H and FIG. 9 panels E-H). However, anti-spike IgG levels as quantified by CBA were much more highly correlated with the levels of FcγRIIIa signaling (R2=0.66) (FIG. 2 panel F) compared to anti-spike titers obtained by ELISA (R2=0.48) (FIG. 2 panel H). Altogether, these data show a strong positive correlation between anti-spike IgG titers, the levels of FcγR activation, and COVID-19 severity in non-hospitalized individuals.

### EXAMPLE 4

### COVID-19 Disease Severity is Correlated With Higher Anti-Spike IgG Cross-Reactivity Against Other Betacoronaviruses (B-CoVs)

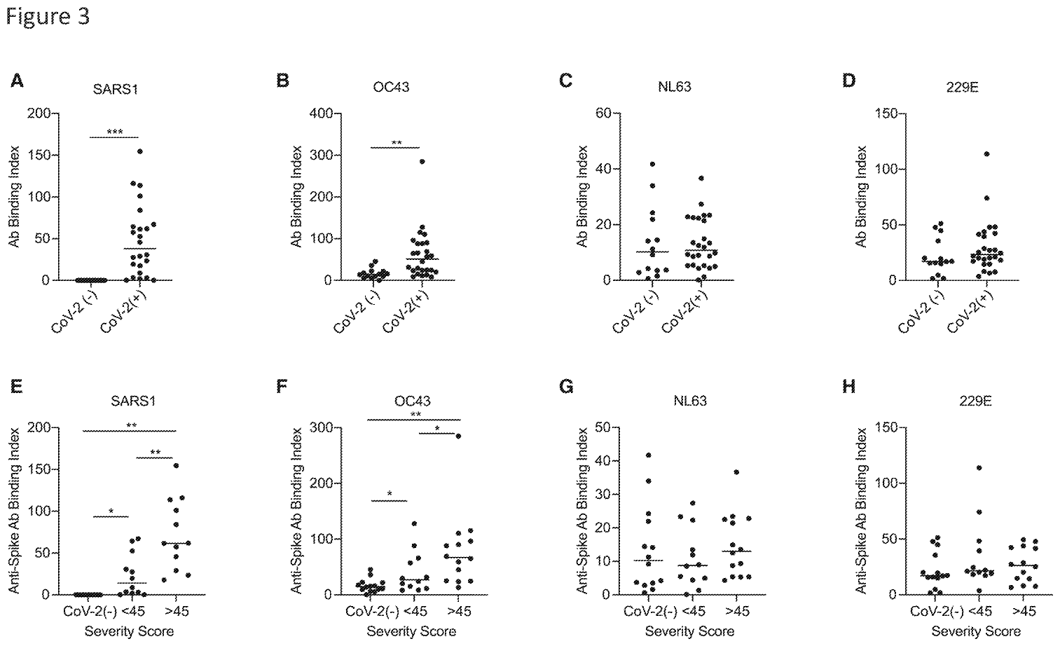

To determine whether humoral recall responses against shCoVs may contribute to SARS-CoV-2 anti-spike IgG responses, we measured the levels of anti-spike IgG binding against the spike of a seasonal B-CoV, OC43, as well as two alphacoronaviruses (α-CoVs), NL63 and 229E. In addition, we examined the level of cross-reactivity of SARS-CoV-2 convalescent IgG against the spike protein of SARS-CoV-1 (SARS1). Since we observed more sensitive detection of anti-spike IgG binding using CBA versus ELISA, we quantified the levels of hCoV anti-spike IgG binding using CBA. We observed that the majority of SARS-CoV-2 convalescent donors possessed IgG, which recognized the SARS1 spike protein (FIG. 3 panel A). Moreover, IgG recognition of the OC43 spike was significantly elevated in SARS-CoV-2 convalescent donors as compared to SARS-CoV-2-naive donors (FIG. 3 panel B). In contrast, IgG binding to α-CoV spike proteins NL63 and 229E was not significantly different among SARS-CoV-2-convalescent and-naive donors (Figure panel C and panel D). This finding suggests that IgG responses against SARS-CoV-2 did not boost IgG responses against α-CoVs. When further explored in relation to disease severity, IgG from severe donors possessed higher cross-reactivity to the spike protein of SARSI and OC43 as compared to mild donors (FIG. 3 panel E and panel F). These findings show that infection with SARS-CoV-2 induces IgG that are cross-reactive with other β-CoVs, and higher levels of cross-reactive IgG correlate with more severe disease.

### EXAMPLE 5

### Immunodominant Regions With High OC43 Sequence Identity Differentially Correlate With COVID-19 Severity

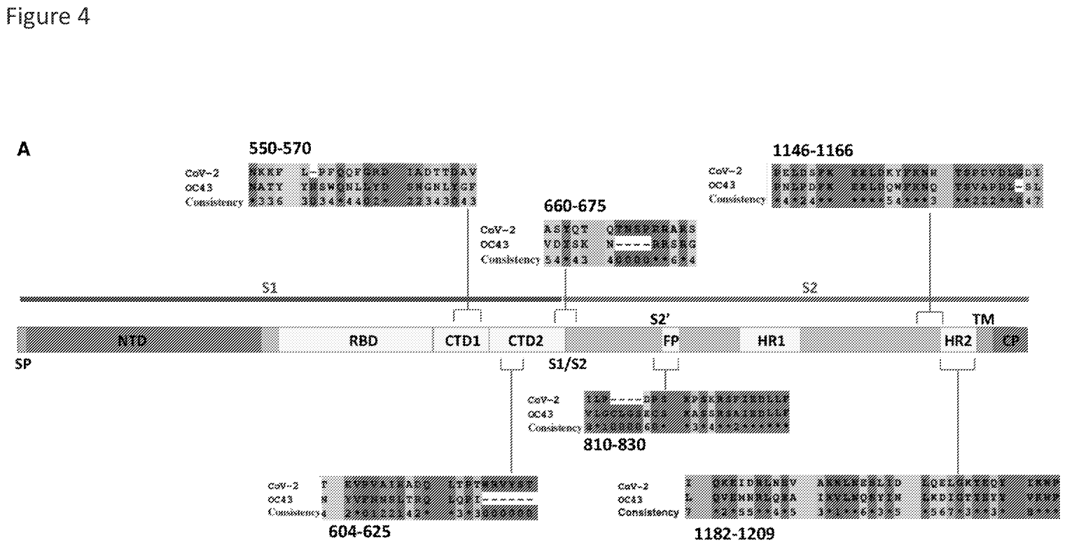

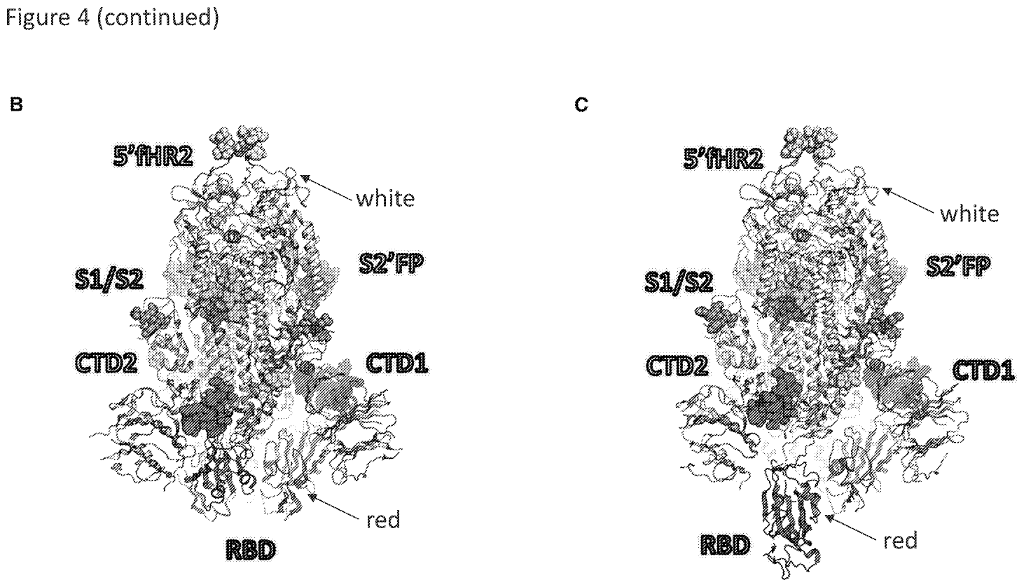

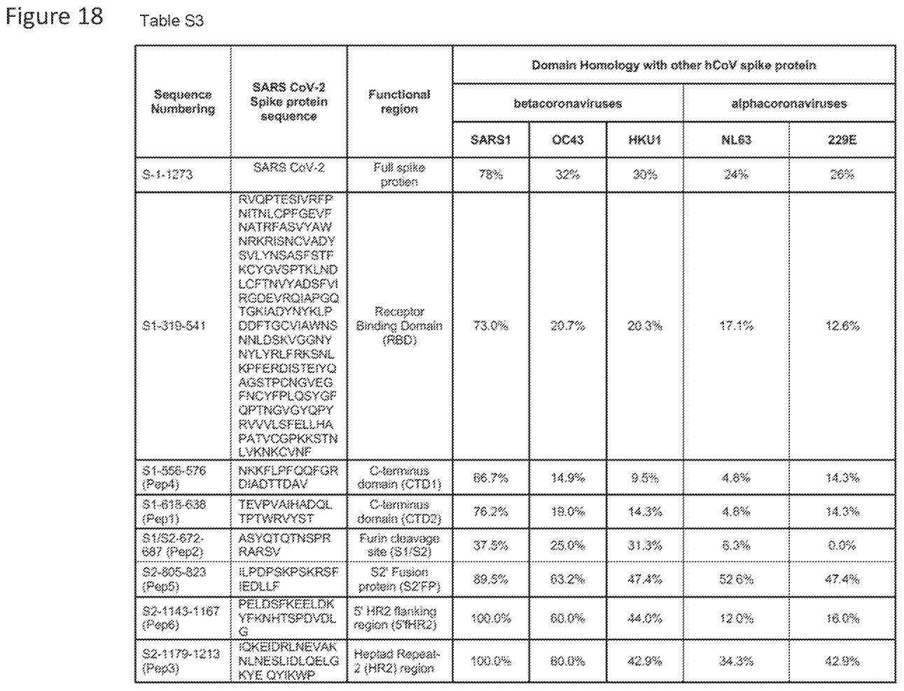

To explore how epitope targeting relates to infection severity in non-hospitalized individuals, we screened our cohort against a panel of immunodominant SARS-CoV-2 peptides that possessed high identity with shCoVs (Table S3 as shown in FIG. 18), in particular β-CoV OC43 (FIG. 4). To contrast these conserved regions, we also screened the levels of IgG-targeting immunodominant regions that possessed little identity with shCoVs. In addition, we quantified the levels of IgG targeting the RBD region, since Abs targeting this region mediate potent SARS-CoV-2 neutralization in vitro and in vivo (Rogers et al., 2020; Wang et al., 2020b; Wec et al., 2020; Zost et al., 2020).

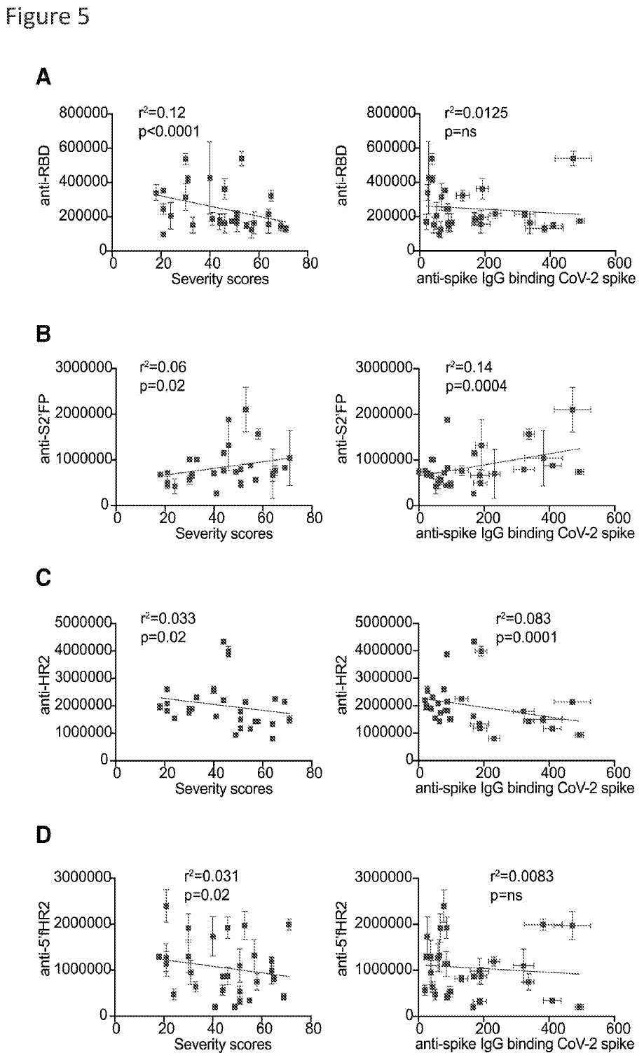

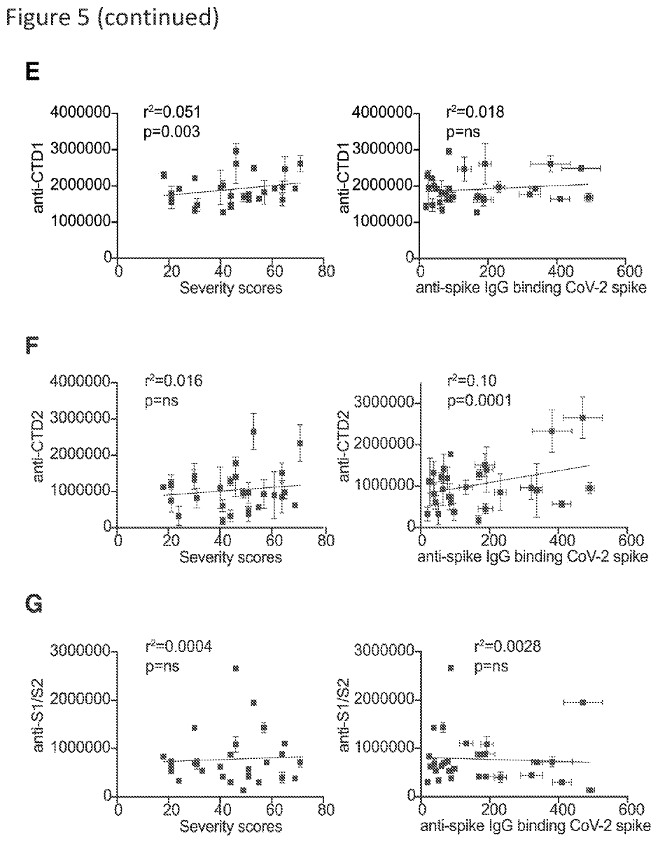

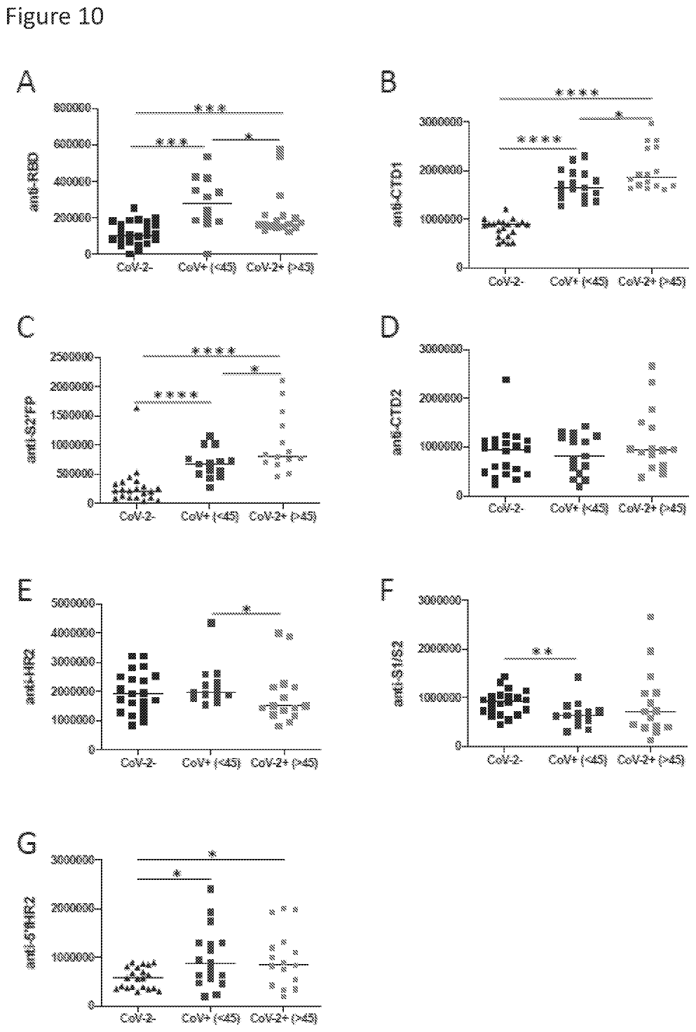

To quantify the levels of IgG targeting these regions, we developed a luciferase-based ELISA to allow for the sensitive detection of IgG binding. Using this assay, we observed a significant inverse correlation between the levels of anti-RBD IgG and overall severity scores among convalescent donors (p<0.0001) (FIG. 5 panel A). In comparing donors with mild versus severe disease, we observed significantly higher levels of anti-RBD IgG targeting in donors with mild disease (FIG. 10 panel A). Next, we quantified the levels of IgG targeting of three representative immunodominant peptides with high sequence identity to shCoVs. We observed a significant positive correlation between S2′FP region IgG-binding levels and severity (FIG. 5 panel B).

In contrast, we observed a significant inverse correlation with the levels of IgG targeting the HR2 region and severity (FIG. 5 panel C), with mild donors possessing higher IgG targeting the HR2 region, as compared to severe donors (FIG. 1 panel E). We also observed that IgG targeting of the HR2 region was inversely correlated with overall anti-spike IgG titers (FIG. 5 panel C). Lastly, we examined IgG targeting of a conserved region located just upstream of the HR2 domain, also known as the 5′ flank HR2 (5′fHR2). Similar to direct targeting of the HR2 region, the levels of IgG binding in the 5′fHR2 region were also inversely correlated with severity (FIG. 5 panel D); however, IgG levels were not inversely correlated with overall anti-spike IgG titers (FIG. 5 panel D).

Next, we quantified the levels of IgG-targeting immunodominant regions that were not highly conserved with OC43 or other shCoV, which include the novel furin cleavage site at the S1/S2 junction and two other regions located within the C-terminal domain (CTD) just downstream of the RBD region (FIG. 4). In screening these regions, we observed a significant positive correlation with the levels of CTD1 targeting and severity (FIG. 5 panel E), but observed no correlation between IgG targeting of the CTD2 or S1/S2 regions and severity (FIG. 5 panels F and G). In comparing binding to these OC43 non-conserved regions and overall anti-spike levels, we detected a significant correlation between IgG targeting of CTD2 and higher anti-spike IgG levels (FIG. 5 panel F), but not with any other regions (Figure panels E and G).

### EXAMPLE 6

### Severe Non-Hospitalized COVID-19 Correlates With High FcγR Activation and IgG Targeting of the Spike Protein S2″ Fusion Protein Site

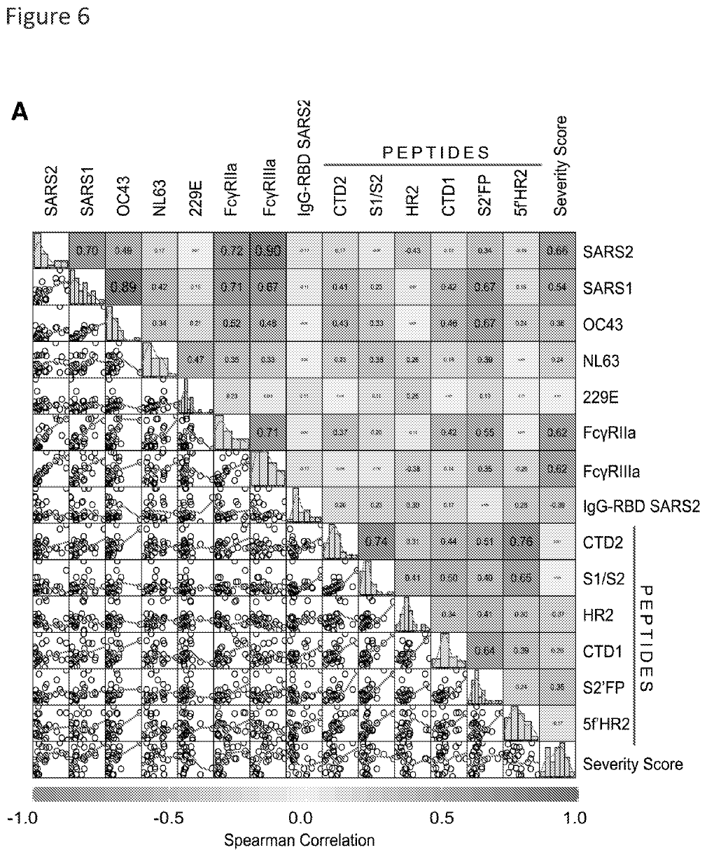

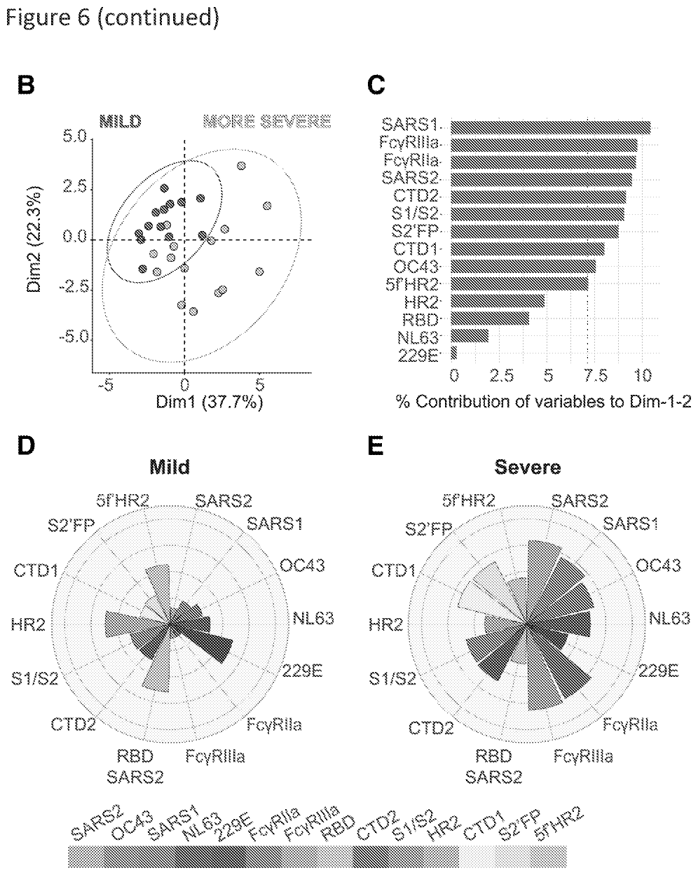

To gain insight into the associations between all of the Ab features tested and disease severity, we performed a Spearman's chart correlation on all of the variables analyzed (FIG. 6 panel A). Through this correlation analysis, we observed that individuals with more severe COVID-19 were characterized by higher anti-SARS-CoV-2 spike IgG (R=0.66), higher IgG cross-reactivity to B-CoVs (SARS1 R=0.54; OC43 R=0.36), and higher proinflammatory FcγR activation (R=0.62), along with higher levels of IgG targeting the CTD1 (R=0.26) and S2′FP regions (R=0.35), as compared to mild donors (FIG. 6 panel A). These results reaffirm the significance of all the observation described in this disclosure above (FIG. 1, FIG. 2, FIG. 3, FIG. 4, FIG. 5). Notably, higher severity scores were inversely correlated with the levels of IgG targeting the RBD and HR2 regions, suggesting that in severe individuals, there is a relative lack of IgG targeting these previously described SARS-CoV-2 neutralizing regions. While we observed that both FcγRIIa and FcγRIIIa activation were positively correlated with β-CoV cross-reactivity, the levels of FcγRIIa signaling were more highly correlated with IgG targeting the CTD1 (R=0.42) and S2′FP (R2=0.55) regions, as compared to FcγRIIIa signaling (CTD1 R=0.14; S2′FP R=0.35).

To further explore the contribution of different IgG features, we performed a principal-component analysis (PCA) using all convalescent donors (FIG. 6 panels B and C). The results of this analysis showed that two dimensions explain the 60% of variance, and the severe individuals are clearly separated according to dimension 1 (37.7% of variance), with a response focused on both FcγRIIa and FcγRIIIa signaling, β-CoV anti-spike IgG (including SARS-CoV-2), and IgG targeting of CTD1, CTD2, and S2′FP regions. Interestingly, the individuals with more severe COVID-19 were highly represented on this dimension, suggesting that cross-reactive IgG responses against β-CoVs, which included the induction of high FcγRIIa and FcγRIIIa activation that targeted CTD1, CTD2, and S2′FP regions, are predictive of severe disease. In contrast, mild individuals possessed different IgG responses, with lower levels on the features within dimension 1, coupled with higher representation of dimension 2 (22.3% of variance), which was primarily represented by IgG targeting of the RBD, S1/S2 furin site, HR2, and 5′fHR2 regions. This analysis suggests a differential course of disease when the IgG response is directed against different regions of the SARS-CoV-2 spike.

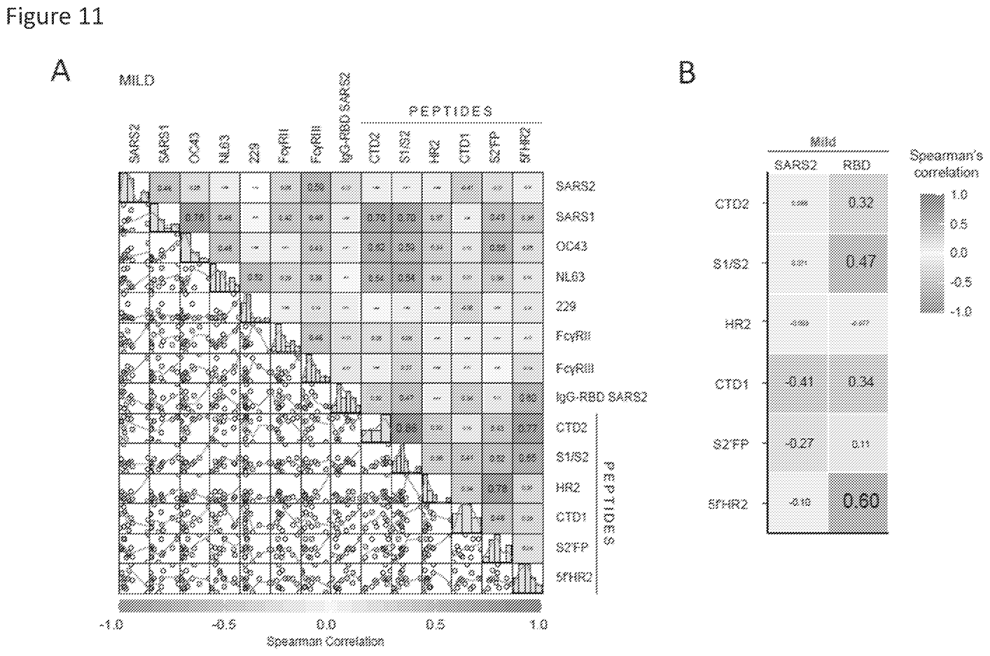

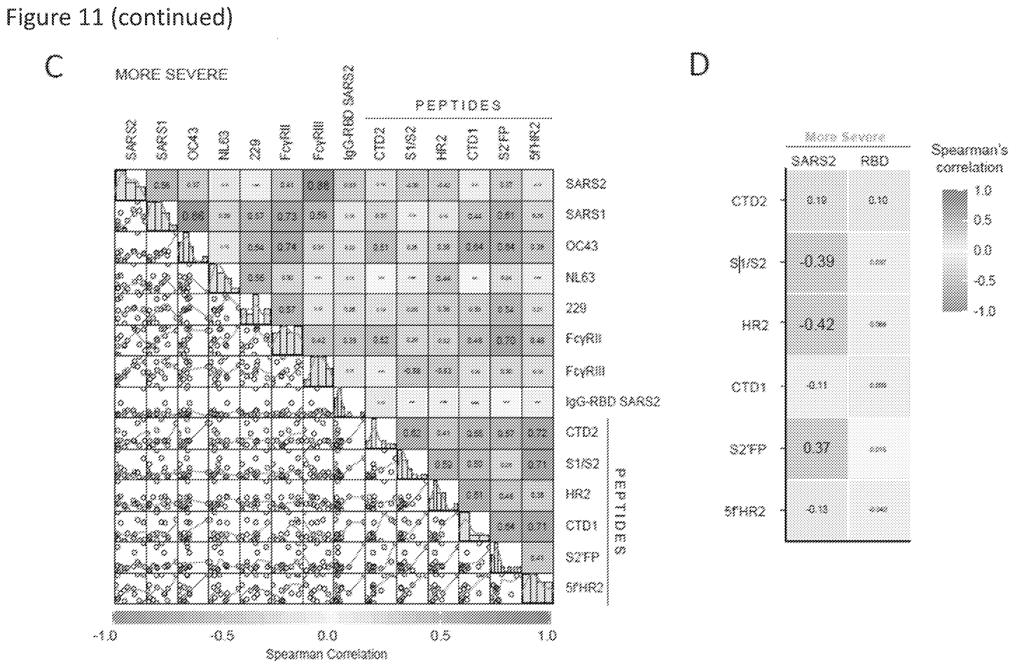

Next, we conducted a Spearman's chart correlation using either mild or severe donor groups (FIGS. 11A and 11C). We observed that FcγRIIa activation was highly correlated with targeting of CTD1, CTD2, and S2′FP regions in severe individuals (FIG. 11 panel C), but no correlation between FcγRIIa activation and IgG targeting of immunodominant regions in mild individuals (FIG. 11A). In only examining the anti-spike region IgG-targeting profiles, we observed that individuals with milder infection possessed anti-spike IgG levels that were inversely correlated with the levels of S2′FP (R=−0.27) and CTD1 (R=−0.41) region IgG targeting (FIG. 11B). The converse was observed in severe profiles, in which total anti-spike IgG levels were inversely correlated with IgG targeting the S1/S2 (R=−0.39) and HR2 (R=−0.42) regions and positively correlated with IgG targeting the S2′FP (R=0.37) region (FIG. 11D). We did not observe a relationship between anti-RBD levels and HR2 region targeting (R=−0.077) in mild individuals.

To further elucidate the differences between the overall profiles of mild and severe individuals, the mean of Z score values for each Ab feature was calculated and represented on a polar plot graph (FIGS. 6D and 6E). In comparing the relative targeting of α- versus β-CoVs, we observed that mild individuals expressed higher Z scores for IgG targeting α-CoVs, with the inverse being true for severe profiles. In support of the PCA analysis, the polar plot shows that individuals with mild disease had lower FcγRIIa and FcγRIIIa enrichment scores (FIG. 6D), which was coupled with relative lower enrichment of IgG targeting the CTD1 and S2′FP regions, both of which were significantly correlated with elevated FcγR activity in severe individuals (FIG. 6D). Crucially, despite severe donors possessing higher overall levels of anti-spike IgG, the enrichment scores for RBD IgG-targeting were higher in mild donors (FIGS. 6D and 6E), suggesting an enrichment in neutralizing IgG. In severe individuals, the inverse was observed: IgG targeting CTD1 and S2′FP regions was enriched, and HR2 region and 5′fHR2 targeting was decreased, relative to total anti-spike IgG (FIG. 6E). Taken together, these data suggest that the efficacy of IgG responses and corresponding disease severity is highly dependent on the specific epitopes targeted in the SARS-CoV-2 spike protein, which in turn may be influenced by prior shCoV exposure.

### EXAMPLE 7

### The Ratio of HR2/S2′FP IgG Targeting Correlates With Disease Severity in Non-Hospitalized Convalescent and ICU-Hospitalized Patients

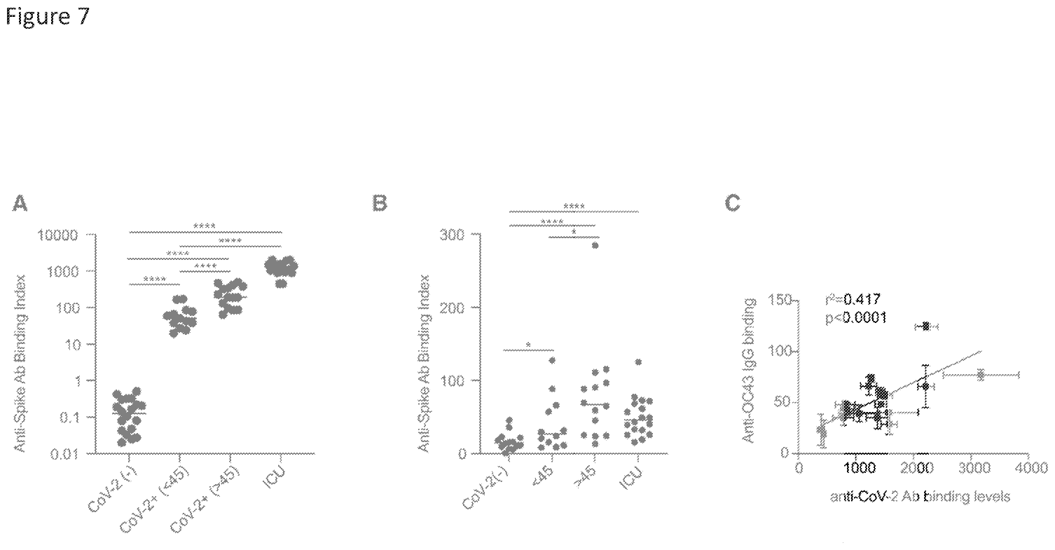

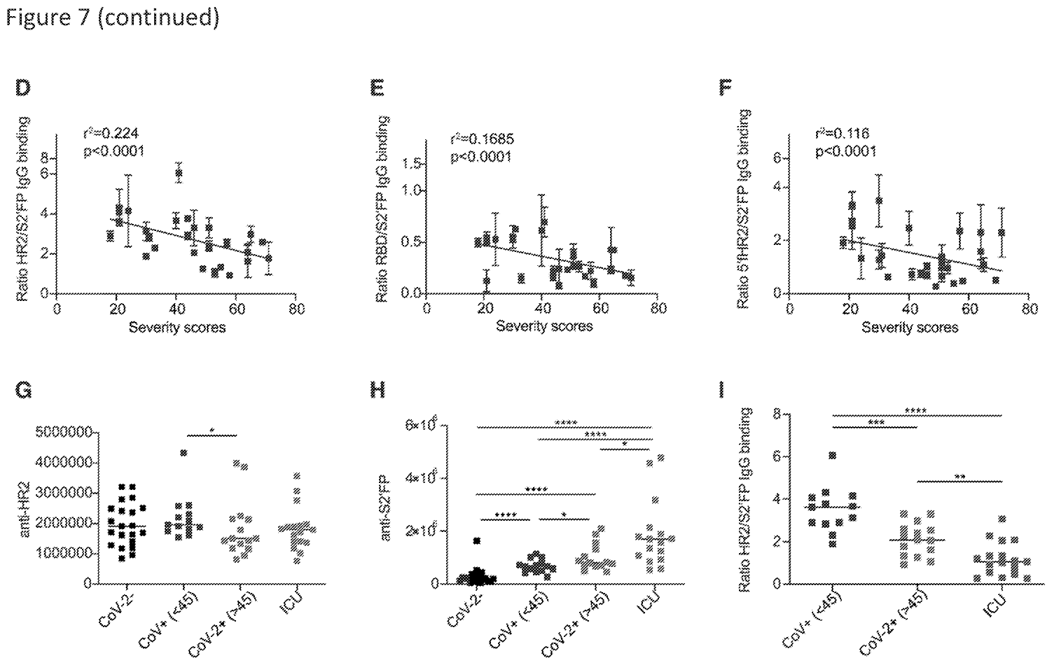

Having established that IgG targeting of the SARS-CoV-2 spike protein was predictive of mild versus severe disease in non-hospitalized patients (FIG. 6), we examined whether this Ab-targeting profile was recapitulated in ICU-hospitalized COVID-19 patients. For this analysis, we obtained a cohort of 17 patients hospitalized in the ICU with COVID-19 during the first waves of the COVID-19 pandemic in 2020. We quantified the levels of IgG directed against the SARS-CoV-2 spike protein in ICU-hospitalized individuals using our cell-based assay. We observed a range of anti-spike IgG levels that were significantly higher than the anti-spike IgG levels detected in non-hospitalized donors (FIG. 7A). Next, we examined the levels of anti-OC43 IgG in ICU patients. Interestingly, while the anti-OC43 IgG levels were significantly higher in ICU patients as compared to SARS-CoV-2-naive donors, ICU patient anti-OC43 IgG levels were in the same range as those detected in non-hospitalized convalescent patients (FIG. 7B). In comparing the levels of SARS-CoV-2 versus OC43 IgG-binding levels in ICU patients, we detected a positive correlation between total anti-SARS-CoV-2 and anti-OC43 spike titers (FIG. 7C).

To define the IgG-targeting profile that best segregated mild versus severe disease, we individually examined the ratios of the three epitopes (RBD, HR2, 5′fHR2) that were associated with mild disease over the epitope (S2′FP) that was most highly associated with severe disease in non-hospitalized donors. Calculating the IgG-targeting ratio of each donor normalizes against the higher anti-SARS-CoV-2 IgG titers detected in more severe patients. In plotting the ratio of RBD/S2′FP, HR2/S2′FP, and 5′fHR2/S2′FP versus severity scores, we observed that all of these IgG-targeting ratios inversely correlated with higher severity scores (FIGS. 7D-7F); however, the ratio of HR2/S2′FP displayed the highest R2 values (R2=0.2239), compared to either RBD/S2′FP (R2=0.1685) or 5′fHR2/S2′FP (R2=0.1158). We next examined the levels of IgG targeting the HR2 and S2′FP regions using IgG from the ICU-hospitalized COVID-19 cohort. We observed that ICU patients possess similar levels of IgG targeting the HR2 region as compared to non-hospitalized convalescent donors (FIG. 7G). In contrast, we detected significantly higher levels of IgG targeting the S2′FP region in ICU-hospitalized patients, as compared to non-hospitalized individuals (FIG. 7H). In calculating HR2/S2′FP IgG-binding ratios, we observed significantly lower ratios in ICU patients as compared to non-hospitalized individuals (FIG. 7I).

### EXAMPLE 8

### Experimental Model and Subject Details

### Human Subjects and Samples

Demographic data (age, sex, COVID-19 RT-PCR status), and collection site and date of the serum/plasma donors studied herein are described in Table S1 as shown in FIG. 16. COVID-19 convalescent donor samples (n=28) and COVID-19 negative donor (n=20) were collected at Ichor Biologics facility in New York City, USA. SARS-CoV-2 negative donors had no history of positive SARS-CoV-2 PCR test or serology test, and had not experienced any symptoms of infection in at least the 5 months prior to blood collection. From convalescent donors, 12 were male and 16 were female, while across naïve donors, 12 were female and 8 were male. As displayed in Table S1 as shown in FIG. 16, the median age of convalescent and naïve groups of donors was 38 years old, and the median of days since the onset of disease to blood draw were 43 and 55.5, respectively. Blood type diversity and underlying health conditions were also presented in Table S1 as shown in FIG. 16, showing a broad range of biological contexts among the cohort. Blood samples were collected after obtaining signed informed consent in accordance with institutionally approved IRB protocols (SSV ORD-2260). Convalescent COVID-19 patient samples were collected from donors 2-10 weeks after the onset of symptoms. A history of COVID-19 symptoms and symptom intensities, along with current medications and history of pre-existing conditions, were collected through participant questionnaires completed at the time of blood draw. The reported symptoms were summarized in Table S2 as shown in FIG. 17, including the average severity and duration of each one. This information was used to calculate a severity score and to cluster donors into two categories: mild and more severe (n=13, n=15, respectively). More severe individuals, with severity scores over 45, and also mild individuals, with severity scores bellow 45, did not require hospitalization. Also, every donor recovered from disease. In addition, 17 individuals were recruited after being diagnosed with a severe course of disease which required invasive ventilation at intensive care units (ICU) from southern Chile (Hospital Base San José, Osorno and Valdivia). Their samples were obtained seven days after recruitment and signing of informed consents in accordance with IRB Servicio Salud Valdivia ORD number 226. Lithium heparin-coated tubes were used for blood collection and plasma was isolated using Ficoll-Hypaque (GE Healthcare; 17-1440-03) in accordance with manufacturer's instructions. Polyclonal IgG was isolated from 200 μL of donor plasma using a protein A/G spin column kit, followed by desalting using Zeba spin columns according to manufacturer's instructions (ThermoFisher Scientific; 89892). IgG yields were quantified using an Easy-Titer Human IgG Assay Kit (ThermoFisher Scientific; 23310). Remaining deidentified plasma samples were aliquoted and stored at −80° C.

### Cell Lines

293T cells: This female cell line was grown in Dulbecco's modified Eagle's medium (DMEM) (Cytiva) supplemented with 10% cosmic calf serum (CCS) (Hyclone), Lglutamine (Corning), and Penicillin-Streptomycin (Hyclone).

FcγRIIa and FcγRIIIa, CD4+ Jurkat reporter cell lines: This male cell line was grown in RPMI-1640 medium (Corning) supplemented with 10% fetal bovine serum (FBS) (Hyclone), L-glutamine (Corning) and Penicillin-Streptomycin (Hyclone).

### Method Details

### SARS-CoV-2 Spike-ELISA

High binding capacity 96-well plates (Nunc) were washed and coated with 50 μL per well of 2 μg/mL of recombinant spike protein (Sino Biological; 40589-V08B1-B), diluted in 0.1% BSA, 0.05% Tween20 TBST ELISA wash buffer (ThermoFisher Scientific; N503). Plates were coated for 2 h at room temperature while shaking at 500 rpm on a Benchmark Orbishaker™. Plates were then washed twice with ELISA wash buffer to remove any excess unbound spike protein and blocked with 2% BSA (ThermoFisher Scientific; 37,525) in ELISA wash buffer overnight at 4° C. After overnight blocking step, plates were washed twice and incubated with 5 μg/mL of donor-derived polyclonal IgG for 1 h at room temperature. After incubation, plates were washed three times and incubated for 30 min at room temperature with cross-absorbed goat anti-human IgG-horseradish peroxidase (HRP)-conjugated secondary antibody (ThermoFisher Scientific; A18811) diluted to a 1:2500 dilution in ELISA wash buffer. After being washed again twice, 100 μL of TMB substrate solution was added to each well for 15 mins and then 100 μL of 0.18M H2SO4 (ThermoFisher Scientific; N600) was added to stop the reaction. The optical density at 450 nm (OD450) was measured using a BioTek Powerwave HT plate reader using Gen5 software. Assay background was established using anti-human secondary Ab alone without donor IgG, which was subtracted from OD values of all samples tested.

### Anti-Spike Protein IgG Determination Using a Cell-Based Assay

To quantify the levels of IgG binding various coronavirus spike proteins, 293T cells were transfected with SARS-CoV-2 (Sino Biological; VG40589-CF) (Genbank: YP_009724390.1), SARS-CoV-1 (Sino Biological; VG40150-CF) (Genbank: AAP13567.1), OC43 (Sino Biological; VG40607-CF) (Genbank: AVR40344.1), NL63 (Sino Biological; VG40604-CF) (Genbank: APF29071.1), or 229E (Sino Biological; VG40605-CF) (Genbank: APT69883.1) spike protein expression vectors. For this assay, 2×106 293T cells were plated in 10 cm plates and incubated at 37° C. overnight. The next day, 4 μg of coronavirus spike expression vectors were transfected into 293T cells using Polyjet™ transfection reagent (SignaGen; SL 100688) according to manufacturer's instructions. After 48 h, 1×105 293T cells were plated per well into round bottom 96-well plates. Cells were then washed and incubated with 10 μg/mL of convalescent donor-derived IgG or negative donor control IgG and incubated at 4° C. for 45 min. After primary Ab incubation, IgG opsonized cells were washed and incubated with 3 μg/mL of an APC-conjugated anti-human total IgG secondary Ab (Invitrogen, catalog A21445) at 4° C. for 25 mins. Cells were then washed again with PBS and LIVE/DEAD™ Fixable Violet Stain (Invitrogen; L34964A) was used to stain cells for 10 min in the dark at RT. Lastly, cells were washed twice and fixed with 1.0% paraformaldehyde in PBS and analyzed by flow cytometry (BD LSRFortessa X-20). The data were quantified using Flow Jo software (Tree Star, Inc). The IgG-binding index was calculated by multiplying the percentage of anti-spike IgG positive cells by the median fluorescent intensity (MFI) of APC signal, as normalized to the average MFI of negative control IgG. To ensure that the relative differences between patient-derived IgG were maintained, all IgG were tested in parallel on the same day for each replicate.

### Fc-Gamma Receptor Signaling Assay

FcγRIIa and FcγRIIIa signaling was assessed using a reporter cell co-culture system that we have previous used to assess FcγR signaling in response to viral antigens ([59, 60]). For this assay, 293T cells are transfected with SARS-CoV-2 spike expression vector and co-cultured with either a FcγRIIa, or FcγRIIIa, CD4+ Jurkat reporter cell line, which expresses firefly luciferase upon FcγR activation. For this assay, 1×105 SARS-CoV-2 spike-expressing 293T cells were plated in each well of a 96-well round bottom plate. The cells were then preincubated with a 5-fold dilution series of convalescent donor-derived IgG starting at a maximum concentration of 25 μg/mL. IgG opsonized 293T cells were then co-cultured with FcγRIIa or FcγRIIIa reporter cells at a 2:1 reporter-to-target cell ratio for 24 h at 37° C. After 24 h, all cells were lysed with cell lysis buffer (Promega; E1531), and the levels of firefly luciferase activity determined using a luciferase assay kit according to manufacturer's instructions (Promega; E1500). To quantify background (i.e., IgG activation-independent) luciferase production, reporter cells were co-cultured with the spike-expressing 293T cells in the absence of any IgG. Background levels were subsequently subtracted from the signal to yield IgG-specific activation in relative light units (RLUs). Luminescence was measured on a Cytation 3 image reader using Gen5 software.

Immunodominant epitope IgG-binding assay. For this assay, N-terminus biotinylated peptides were synthesized by Genscript. N-terminal GSGS (SEQ ID NO:34) linker sequence was added to all peptide sequences. The RBD peptide contained a C-terminal avitag (GLNDIFEAQKIEWHE (SEQ ID NO:35)), for biotinylation via BirA enzyme; a Protein C tag (EDQVDPRLIDGK (SEQ ID NO:36)), and a polyhisidine tag (HHHHHHHHHH (SEQ ID NO:37)), to enable immobilized metal affinity chromatography purification. Lyophilized peptides and RBD were initially resuspended in DMSO and then used to make 5 μg/mL working dilutions in TBST ELISA wash buffer. Pierce™ white streptavidin-coated high binding capacity 96-well binding plates (ThermoFisher Scientific; 15502) were washed twice with ELISA wash buffer and coated with 5 μg/mL of biotinylated peptides at room temp. Plates were coated for 2 h while shaking at 500 rpm on a Benchmark Orbi-Shaker™. After incubation, plates were washed three times and blocked with 2% BSA blocking solution diluted in wash buffer and incubated at 4° C. overnight. After incubation, plates were washed three times and incubated with 5 μg/mL of donor-derived IgG for 1 h at room temp. After primary IgG incubation, plates were washed 3 times and incubated for 25 mins with 100 μL of Invitrogen™ cross-absorbed, F (ab′)₂, goat anti-human IgG secondary Ab (ThermoFisher Scientific; A24470) diluted 1:2500 in ELISA wash buffer. Plates were then washed again three times and developed using a SuperSignal™ ELISA Pico Chemiluminescent Substrate (ThermoFisher; Cat #37069) and the level of luminescence detected using a Cytation 3 image reader luminometer using Gen5 software.

### Quantification and Statistical Analysis

Statistical and data analyses were performed using GraphPad Prism 8.4.3, R 4.0.4, and R Studio 1.4.1103. Graphs were generated in Prism and R Studio and statistical differences between two groups were calculated by Mann-Whitney U-test. Statistical significance was defined as *p<0.05; **p<0.01; ***p<0.001, and ****p<0.0001. Scatter plots, bar graphs, heatmaps, and polar plots were visualized with ggplot2 (v3.3.3 R Studio). Correlation analysis (in FIGS. 7 and 11) were performed using the R package “correlation” (v0.6.0) in R Studio. Polar plots represent the value of different variables normalized to the Z-score of data. Each variable was mean-centered and then divided by the standard deviation of the variable to ensure each variable had zero mean and unit standard deviation. Unsupervised principal components analysis (PCA) was performed in R. The completed data were scaled to unit variance using FactoMineR (v2.4 R studio). The PCA results were extracted and visualized using factoextra (v1.0.7 R Studio). Outlier exclusion was performed using Prism. n and N values are mentioned at figure captions.

### Discussion of Examples

It will be recognized from this disclosure that, early in the pandemic, it was largely assumed that the adaptive immune system treated SARS-CoV-2 as a novel pathogen, despite the significant sequence overlap of the virus with shCoVs. Although subsequent studies examined seasonal coronavirus immune recall, immunodominant epitopes, and COVID-19 severity individually, none looked at all three factors together. Accordingly, the role of memory Abs in disease pathogenesis has been unclear and clinically disregarded. Researchers have noted an unusual aspect of COVID-19 disease course; namely, some patients show IgG within days of contracting SARS-CoV-2 (Liu et al., 2020; Zhang et al., 2020; Zhao et al., 2020). This is in contrast to the 7-14 days that is usually required for naive B cells to become activated, class switch, and begin producing IgG (Murphy et al., 2012). Notably, these early anti-spike IgG responses are associated with disease severity, not protection. Higher anti-spike IgG titers, along with elevated levels of proinflammatory cytokines, correlate with more severe disease in hospitalized individuals (Young et al., 2020; Zhang et al., 2020). This disclosure demonstrates a significant positive correlation between anti-SARS-CoV-2 spike IgG levels and disease severity in a cohort of non-hospitalized convalescent individuals with mild to severe COVID-19, as well as even higher anti-spike IgG titers in ICU patients. Along with higher anti-spike IgG levels, we detected that IgG cross-reactivity to other B-CoVs (SARSI and OC43) is positively correlated with disease severity. Considering the ubiquitous seroprevalence of shCoVs (Gorse et al., 2010)—4%-27% of the population tests positive for any given shCoV at any given time (Khan et al., 2021) it is a realistic possibility that early, high levels of cross-reactive anti-spike IgG arise from recall memory responses against shCoVs, such as OC43. The role of these cross-reactive recall Abs has been previously unclear, and previous studies conflict as to whether they possess neutralizing activity against SARS-CoV-2. In addition to higher levels of anti-spike IgG and cross-reactive IgG, we also detected higher levels of anti-spike IgG-mediated FcγRIIa and FcγRIIIa signaling in severe patients in the non-hospitalized cohort. We initially hypothesized that any shCoV recall responses amplified during acute SARS-CoV-2 infections would favor S2—and not the novel RBD-containing S1—subunit targeting inefficient non-neutralizing Ab responses that would lead to uncontrolled virus replication (similar to original antigenic sin), elevated inflammation, and worse outcomes. However, we detected no uniform association between high sequence identity and severity. Instead, we observed that IgG targeting of two highly conserved regions (HR2 region, 5′fHR2) was significantly correlated with milder infections, while targeting of another conserved region (S2′FP) was correlated with more severe infections. All three of the immunodominant conserved regions studied were correlated with disease severity, whether inverse or positive. In contrast, only one of the three non-homologous regions had a significant relationship with disease course. Examining the IgG profiles of convalescent individuals in the context of functional regions, we observed that IgG targeting of RBD, HR2, and HR flanking regions significantly correlated with lower severity scores and, in the case of HR2, also correlated with lower levels of anti-spike IgG. This latter finding suggests that humoral responses with a higher proportion of IgG targeting the HR2 region may be highly efficient at controlling infection, since lower anti-spike titers were independently associated with milder infections. Altogether, these data show that IgG targeting in and around the HR2 region may induce potent neutralization of SARS-CoV-2. Furthermore, in comparing the levels of RBD and immunodominant epitope targeting in mild donors, we observed no correlation between RBD and HR2 region IgG targeting, despite both being correlated with mild disease. This raises the possibility of at least two distinct neutralization profiles, possibly driven by whether humoral responses are predominately recall or de novo. We also observed IgG targeting of the non-conserved CTD2 region and the conserved S2′FP region significantly correlated with higher severity scores in convalescent donors. In examining only severe donor profiles, we resolved that they were predominantly characterized by higher levels of IgG targeting S2′FP. Functionally, in the context of virus entry, the fusion protein region inserts into the host cell membrane, facilitating virus fusion into host cells (Harrison, 2015; Kawase et al., 2019). In contrast to HR2 and 5′fHR2, Abs targeting the S2′FP and CTD regions have been less well characterized. While we did not examine IgG neutralization activity, we did examine the capacity of IgG to induce FcγR activation, a prerequisite for inducing non-neutralizing effector functions such as proinflammatory cytokine release. In this analysis, we identify that increased FcγR signaling is highly correlated with higher IgG targeting of the CTD1 and S2′FP regions and more severe COVID-19. CTD1 had a lower R2 value than S2′FP, possibly indicating weaker association. In contrast, the levels of HR2 or 5′HR2 IgG were not correlated with FcγR signaling. These findings suggest that IgG targeting of the S2′FP and/or CTD1 region could indicate sites of enhanced FcγR activation. In only examining the severe convalescent donors, we observed that severe responses were not solely characterized by higher levels of S2′FP targeting, but also the absence of IgG targeting the S1/S2 furin site and HR2 regions. Similarly, milder IgG profiles were not only characterized by higher levels of RBD and/or HR2 targeting, but also lower proportions of CTD1 and S2′FP targeting in relation to overall anti-spike titers. These findings strongly suggest that targeting the S2′FP region, coupled with the absence of anti-RBD and/or anti-HR2 IgG targeting, is linked with severity. S2′FP targeting may be merely inefficient or it may actively cause deleterious ADE or ADI effects. Multivariate PCA analysis showed that profiles with higher levels of IgG targeting of HR2, 5′fHR2, and RBD regions were predictive of mild disease, while higher levels of IgG targeting the S2′FP region were predictive of severe disease in non-hospitalized donors. In these recently convalescent individuals, the ratio of IgG targeting the HR2/S2′FP regions was most tightly correlated with having mild versus severe disease, as compared to the ratios of IgG targeting 5′fHR2/S2′FP or RBD/S2′FP. Specifically, lower ratios of IgG targeting the HR2/S2′FP regions correlated with more severe disease and higher ratios correlated with mild infections. In examining this signature in ICU-hospitalized COVID-19 patients, we observed even lower ratios of IgG targeting the HR2/S2′FP regions, as compared to severe non-hospitalized donors. These data show that HR2/S2′FP IgG targeting ratios may predict COVID-19 severity. Therefore, determining the dominant epitope profile and associated predicted risk of severe disease of an individual could allow earlier interventions with treatments such as monoclonal Ab therapies and antivirals, preventing progression to ARDS. In addition, assessing the humoral profiles induced by vaccination is encompassed by this disclosure. Evidence has emerged that SARS-CoV-2 reinfection is possible after 3-6 months, particularly with emerging variants (Abu-Raddad et al., 2020; Gupta et al., 2020; Selhorst et al., 2020; Tillett et al., 2021). This is in line with shCoVs, in which reinfection is common after 6 months (Edridge et al., 2020). Moreover, the emergence of variants able to evade current vaccines remains an ongoing threat. Both SARS-CoV-2 spike and whole-virus vaccines likely boost both novel and shCoV cross-reactive IgG responses, initially giving some level of protection, but which could lead to higher frequencies of severe breakthrough infections over time if inefficient IgG targeting remains the predominant response. Even if vaccinated with current vaccines, individuals with inefficient IgG profiles (i.e., anti-S2′FP dominant) may require an RBD-specific or HR2-specific vaccine to shift their targeting profiles to induce long-lived protective humoral responses. The disclosure includes use of HR2 as a component of a universal hCoV treatment and vaccine.

The disclosure indicates that humoral memory responses contribute to COVID-19 disease severity, conferring either protection or risk, depending on epitope targeting. This may explain the atypical bimodality of COVID-19 disease severity, an observation that was previously obscured by aggregate data/epitope analysis. It has been challenging to enumerate the underlying factors that accurately predict disease outcome, even among individuals with defined comorbidities. It considered that the instant disclosure helps explain the differential correlations observed in previous studies regarding preexisting cross-reactive immunity to seasonal coronaviruses and disease severity, as well as improve the prediction accuracy of disease outcomes. This epitope-based original antigenic sin immunosurveillance, or OASIS, is therefore suitable for use as a clinical prognostic, offering a novel approach for the prediction of disease severity risk, as well as vaccine efficacy, on a patient-by-patient basis.\

The following reference listing is not an indication that any references are material to patentability:
Alvarez, R.A., Hamlin, R.E., Monroe, A., Moldt, B., Hotta, M.T., Rodriguez Caprio, G., Fierer, D.S., Simon, V., and Chen, B.K. (2014). HIV-1 Vpu antagonism of tetherin inhibits antibody-dependent cellular cytotoxic responses by natural killer cells. J. Virol. 88, 6031-6046. https://doi.org/10.1128/jvi.00449-14.
Alvarez, R.A., Maestre, A.M., Law, K., Durham, N.D., Barria, M.I., Ishii-Watabe, A., Tada, M., Kapoor, M., Hotta, M.T., Rodriguez-Caprio, G., et al. (2017). Enhanced FCGR2A and FCGR3A signaling by HIV viremic controller IgG. JCI Insight 2, e88226. https://doi.org/10.1172/jci.insight.88226.
Anderson, E.M., Goodwin, E.C., Verma, A., Arevalo, C.P., Bolton, M.J., Weirick, M.E., Gouma, S., McAllister, C.M., Christensen, S.R., Weaver, J., et al. (2021). Seasonal human coronavirus antibodies are boosted upon SARSCoV-2 infection but not associated with protection. Cell 184, 1858-1864.e10. https://doi.org/10.1016/j.cell.2021.02.010.
Bournazos, S., Gupta, A., and Ravetch, J.V. (2020). The role of IgG Fc receptors in antibody-dependent enhancement. Nat. Rev. Immunol. 20, 633-643. https://doi.org/10.1038/s41577-020-00410-0.
Chakraborty, S., Gonzalez, J., Edwards, K., Mallajosyula, V., Buzzanco, A.S., Sherwood, R., Buffone, C., Kathale, N., Providenza, S., Xie, M.M., et al. (2021). Proinflammatory IgG Fc structures in patients with severe COVID-19. Nat. Immunol. 22, 67-73. https://doi.org/10.1038/s41590-020-00828-7.
Del Valle, D.M., Kim-Schulze, S., Huang, H.-H., Beckmann, N.D., Nirenberg, S., Wang, B., Lavin, Y., Swartz, T.H., Madduri, D., Stock, A., et al. (2020). An inflammatory cytokine signature predicts COVID-19 severity and survival. Nat. Med. 26, 1636-1643. https://doi.org/10.1038/s41591-020-1051-9.
Edridge, A.W.D., Kaczorowska, J., Hoste, A.C.R., Bakker, M., Klein, M., Loens, K., Jebbink, M.F., Matser, A., Kinsella, C.M., Rueda, P., et al. (2020). Seasonal coronavirus protective immunity is short-lasting. Nat. Med. 26, 1691-1693. https://doi.org/10.1038/s41591-020-1083-1.
Fafi-Kremer, S., Bruel, T., Madec, Y., Grant, R., Tondeur, L., Grzelak, L., Staropoli, I., Anna, F., Souque, P., Fernandes-Pellerin, S., et al. (2020). Serologic responses to SARS-CoV-2 infection among hospital staff with mild disease in eastern France. EBioMedicine 59, 102915. https://doi.org/10.1016/j. ebiom.2020.102915.
Forthal, D.N., and Moog, C. (2009). Fc receptor-mediated antiviral antibodies. Curr. Opin. HIV AIDS 4, 388-393. https://doi.org/10.1097/COH. 0b013e32832f0a89.
Garrido, J.L., Prescott, J., Calvo, M., Bravo, F., Alvarez, R., Salas, A., Riquelme, R., Rioseco, M.L., Williamson, B.N., Haddock, E., et al. (2018). Two recombinant human monoclonal antibodies that protect against lethal Andes hantavirus infection in vivo. Sci. Transl. Med. 10, eaat6420. https://doi.org/10.1126/scitranslmed.aat6420.
Gorse, G.J., Patel, G.B., Vitale, J.N., and O'Connor, T.Z. (2010). Prevalence of antibodies to four human coronaviruses is lower in nasal secretions than in serum. Clin. Vaccine Immunol. 17, 1875-1880. https://doi.org/10.1128/CVI. 00278-10.
Grzelak, L., Temmam, S., Planchais, C., Demeret, C., Tondeur, L., Huon, C., Guivel-Benhassine, F., Staropoli, I., Chazal, M., Dufloo, J., et al. (2020). A comparison of four serological assays for detecting anti-SARS-CoV-2 antibodies in human serum samples from different populations. Sci. Transl. Med. 12, eabc3103. https://doi.org/10.1126/scitranslmed.abc3103.
Gupta, V., Bhoyar, R.C., Jain, A., Srivastava, S., Upadhayay, R., Imran, M., Jolly, B., Divakar, M.K., Sharma, D., Sehgal, P., et al. (2020). Asymptomatic reinfection in 2 healthcare workers from India with genetically distinct severe acute respiratory syndrome coronavirus 2. Clin. Infect. Dis. 73, e2823-e2825. https://doi.org/10.1093/cid/ciaa1451.
Halstead, S.B. (2014). Dengue antibody-dependent enhancement: knowns and unknowns. Microbiol. Spectr. 2. https://doi.org/10.1128/microbiolspec. AID-0022-2014.
Harrison, S.C. (2015). Viral membrane fusion. Virology 479-480, 498-507. https://doi.org/10.1016/j.virol.2015.03.043.
Hoepel, W., Chen, H.J., Allahverdiyeva, S., Manz, X., Aman, J., Biobank, A.U.C.-., Bonta, P., Brouwer, P., de Taeye, S., Caniels, T., et al. (2020). Anti-SARS-CoV-2 IgG from severely ill COVID-19 patients promotes macrophage hyper-inflammatory responses. Preprint at bioRxiv. https://doi.org/10.1101/2020.07.13.190140.
Iwasaki, A., and Yang, Y. (2020). The potential danger of suboptimal antibody responses in COVID-19. Nat. Rev. Immunol. 20, 339-341. https://doi.org/10. 1038/s41577-020-0321-6.
Jaume, M., Yip, M.S., Cheung, C.Y., Leung, H.L., Li, P.H., Kien, F., Dutry, I., Callendret, B., Escriou, N., Altmeyer, R., et al. (2011). Anti-severe acute respiratory syndrome coronavirus spike antibodies trigger infection of human immune cells via a pH- and cysteine protease-independent FcgR pathway. J. Virol. 85, 10582-10597. https://doi.org/10.1128/JVI.00671-11.
Junqueira, C., Crespo, \hat{ }A., Ranjbar, S., de Lacerda, L.B., Lewandrowski, M., Ingber, J., Parry, B., Ravid, S., Clark, S., Schrimpf, M.R., et al. (2022). FcgRmediated SARS-CoV-2 infection of monocytes activates inflammation. Nature. https://doi.org/10.1038/s41586-022-04702-4.
Kam, Y.W., Kien, F., Roberts, A., Cheung, Y.C., Lamirande, E.W., Vogel, L., Chu, S.L., Tse, J., Guarner, J., Zaki, S.R., et al. (2007). Antibodies against trimeric S glycoprotein protect hamsters against SARS-CoV challenge despite their capacity to mediate FcgRII-dependent entry into B cells in vitro. Vaccine 25, 729-740. https://doi.org/10.1016/j.vaccine.2006.08.011.
Kaplonek, P., Wang, C., Bartsch, Y., Fischinger, S., Gorman, M.J., Bowman, K., Kang, J., Dayal, D., Martin, P., Nowak, R.P., et al. (2021). Early cross-coronavirus reactive signatures of humoral immunity against COVID-19. Sci Immunol. 6, eabj2901. https://doi.org/10.1126/sciimmunol.abj2901.
Kawase, M., Kataoka, M., Shirato, K., and Matsuyama, S. (2019). Biochemical analysis of coronavirus spike glycoprotein conformational intermediates during membrane fusion. J. Virol. 93, e00785-e00819. https://doi.org/10.1128/JVI.00785-19.
Keng, C.T., Zhang, A., Shen, S., Lip, K.M., Fielding, B.C., Tan, T.H.P., Chou, C.F., Loh, C.B., Wang, S., Fu, J., et al. (2005). Amino acids 1055 to 1192 in the S2 region of severe acute respiratory syndrome coronavirus S protein induce neutralizing antibodies: implications for the development of vaccines and antiviral agents. J. Virol. 79, 3289-3296. https://doi.org/10.1128/JVI.79. 6.3289-3296.2005.
Khan, T., Rahman, M., Al Ali, F., Huang, S.S.Y., Ata, M., Zhang, Q., Bastard, P., Liu, Z., Jouanguy, E., Be' ziat, V., et al. (2021). Distinct antibody repertoires against endemic human coronaviruses in children and adults. JCI insight 6, e144499. https://doi.org/10.1172/jci.insight.144499.
Ladner, J.T., Henson, S.N., Boyle, A.S., Engelbrektson, A.L., Fink, Z.W., Rahee, F., D'Ambrozio, J., Schaecher, K.E., Stone, M., Dong, W., et al. (2021). Epitope-resolved profiling of the SARS-CoV-2 antibody response identifies cross-reactivity with endemic human coronaviruses. Cell Rep. Med. 2, 100189. https://doi.org/10.1016/j.xcrm.2020.100189.
Lai, S.C., Chong, P.C.S., Yeh, C.T., Liu, L.S., Jan, J.T., Chi, H.Y., Liu, H.W., Chen, A., and Wang, Y.C. (2005). Characterization of neutralizing monoclonal antibodies recognizing a 15-residues epitope on the spike protein HR2 region of severe acute respiratory syndrome coronavirus (SARS-CoV). J. Biomed. Sci. 12, 711-727. https://doi.org/10.1007/s11373-005-9004-3.
Larsen, M.D., de Graaf, E.L., Sonneveld, M.E., Plomp, H.R., Nouta, J., Hoepel, W., Chen, H.J., Linty, F., Visser, R., Brinkhaus, M., et al. (2020). Afucosylated IgG characterizes enveloped viral responses and correlates with COVID-19 severity. Science 371, eabc8378. https://doi.org/10.1126/science.abc8378.
Lee, W.S., Wheatley, A.K., Kent, S.J., and DeKosky, B.J. (2020). Antibodydependent enhancement and SARS-CoV-2 vaccines and therapies. Nat. Microbiol. 5, 1185-1191. https://doi.org/10.1038/s41564-020-00789-5.
Li, Y., Lai, D.-y., Zhang, H.-n., Jiang, H.-w., Tian, X., Ma, M.-l., Qi, H., Meng, Q.-f., Guo, S.-j., Wu, Y., et al. (2020). Linear epitopes of SARS-CoV-2 spike protein elicit neutralizing antibodies in COVID-19 patients. Cell. Mol. Immunol. 17, 1095-1097. https://doi.org/10.1038/s41423-020-00523-5.
Li, Y., Ma, M.L., Lei, Q., Wang, F., Hong, W., Lai, D. Y., Hou, H., Xu, Z.W., Zhang, B., Chen, H., et al. (2021). Linear epitope landscape of the SARSCoV-2 Spike protein constructed from 1,051 COVID-19 patients. Cell Rep. 34, 108915. https://doi.org/10.1016/j.celrep.2021.108915.
Lip, K.M., Shen, S., Yang, X., Keng, C.T., Zhang, A., Oh, H.L.J., Li, Z.H., Hwang, L.A., Chou, C.F., Fielding, B.C., et al. (2006). Monoclonal antibodies targeting the HR2 domain and the region immediately upstream of the HR2 of the S protein neutralize in vitro infection of severe acute respiratory syndrome coronavirus. J. Virol. 80, 941-950. https://doi.org/10.1128/JVI.80.2. 941-950.2006.
Liu, L., Wei, Q., Lin, Q., Fang, J., Wang, H., Kwok, H., Tang, H., Nishiura, K., Peng, J., Tan, Z., et al. (2019). Anti-spike IgG causes severe acute lung injury by skewing macrophage responses during acute SARS-CoV infection. JCI Insight 4, e123158. https://doi.org/10.1172/jci.insight.123158.
Liu, X., Wang, J., Xu, X., Liao, G., Chen, Y., and Hu, C.H. (2020). Patterns of IgG and IgM antibody response in COVID-19 patients. Emerg Microbes Infect. 9, 1269-1274. https://doi.org/10.1080/22221751.2020.1773324.
Long, Q.-X., Liu, B.-Z., Deng, H.-J., Wu, G.-C., Deng, K., Chen, Y.-K., Liao, P., Qiu, J.-F., Lin, Y., Cai, X.-F., et al. (2020). Antibody responses to SARS-CoV-2 in patients with COVID-19. Nat. Med. 26, 845-848. https://doi.org/10.1038/s41591-020-0897-1.
McNaughton, A.L., Paton, R.S., Edmans, M., Youngs, J., Wellens, J., Phalora, P., Fyfe, A., Belij-Rammerstorfer, S., Bolton, J.S., Ball, J., et al. (2021). Fatal COVID-19 outcomes are associated with an antibody response targeting epitopes shared with endemic coronaviruses. Preprint at medRxiv. https://doi. org/10.1101/2021.05.04.21256571.
Mishra, N., Huang, X., Joshi, S., Guo, C., Ng, J., Thakkar, R., Wu, Y., Dong, X., Li, Q., Pinapati, R., et al. (2020). Immunoreactive peptide maps of SARS-CoV-2 and other human coronaviruses. Preprint at bioRxiv. https://doi.org/10.1101/2020.08.13.249953.
Mishra, N., Huang, X., Joshi, S., Guo, C., Ng, J., Thakkar, R., Wu, Y., Dong, X., Li, Q., Pinapati, R.S., et al. (2021). Immunoreactive peptide maps of SARSCoV-2. Commun. Biol. 4, 225. https://doi.org/10.1038/s42003-021-01743-9.
Mohsin, S.N., Mahmood, S., Amar, A., Ghafoor, F., Raza, S.M., and Saleem, M. (2015). Association of FcgRIIa polymorphism with clinical outcome of dengue infection: first insight from Pakistan. Am. J. Trop. Med. Hyg. 93, 691-696. https://doi.org/10.4269/ajtmh.15-0199.
Murphy, K., Travers, P., Walport, M., and Janeway, C. (2012). Janeway's Immunobiology, 8th Edition (Garland Science).
Ng, K.W., Faulkner, N., Cornish, G.H., Rosa, A., Harvey, R., Hussain, S., Ulferts, R., Earl, C., Wrobel, A.G., Benton, D.J., et al. (2020). Preexisting and de novo humoral immunity to SARS-CoV-2 in humans. Science 370, 1339-1343. https://doi.org/10.1126/science.abe1107.
Nguyen-Contant, P., Embong, A.K., Kanagaiah, P., Chaves, F.A., Yang, H., Branche, A.R., Topham, D.J., and Sangster, M.Y. (2020). S protein-reactive IgG and memory B cell production after human SARS-CoV-2 infection includes broad reactivity to the S2 subunit. mBio 11. https://doi.org/10.1128/mBio.01991-20.
Nimmerjahn, F., and Ravetch, J.V. (2010). Antibody-mediated modulation of immune responses. Immunol. Rev. 236, 265-275. https://doi.org/10.1111/j. 1600-065X.2010.00910.x.
Okba, N.M.A., Muller, M.A., Li, W., Wang, C., GeurtsvanKessel, C.H., Corman, V.M., Lamers, M.M., Sikkema, R.S., de Bruin, E., Chandler, F.D., et al. (2020). Severe acute respiratory syndrome coronavirus 2-specific antibody responses in coronavirus disease patients. Emerg. Infect. Dis. 26, 1478-1488. https://doi. org/10.3201/eid2607.200841.
Pincetic, A., Bournazos, S., DiLillo, D.J., Maamary, J., Wang, T.T., Dahan, R., Fiebiger, B.-M., and Ravetch, J.V. (2014). Type I and type II Fc receptors regulate innate and adaptive immunity. Nat. Immunol. 15, 707-716. https://doi.org/10.1038/ni.2939.
Qu, J., Wu, C., Li, X., Zhang, G., Jiang, Z., Li, X., Zhu, Q., and Liu, L. (2020). Profile of immunoglobulin G and IgM antibodies against severe acute respiratory syndrome coronavirus 2 (SARS-CoV-2). Clin. Infect. Dis. 71, 2255-2258. https://doi.org/10.1093/cid/ciaa489.
Ricke, D.O. (2021). Two different antibody-dependent enhancement (ADE) risks for SARS-CoV-2 antibodies. Front. Immunol. 12, 640093. https://doi. org/10.3389/fimmu.2021.640093.
Rogers, T.F., Zhao, F., Huang, D., Beutler, N., Burns, A., He, W.T., Limbo, O., Smith, C., Song, G., Woehl, J., et al. (2020). Isolation of potent SARS-CoV-2 neutralizing antibodies and protection from disease in a small animal model. Science 369, 956-963. https://doi.org/10.1126/science.abc7520.
Selhorst, P., van Ierssel, S.H., Michiels, J., Marie” n, J., Bartholomeeusen, K., Dirinck, E., Vandamme, S., Jansens, H., and Arie” n, K.K. (2020). Symptomatic severe acute respiratory syndrome coronavirus 2 reinfection of a Healthcare worker in a Belgian nosocomial outbreak despite primary neutralizing antibody response. Clin. Infect. Dis. https://doi.org/10.1093/cid/ciaa1850.
Shah, P., Canziani, G.A., Carter, E.P., and Chaiken, I. (2021). The case for S2: the potential benefits of the S2 subunit of the SARS-CoV-2 spike protein as an immunogen in fighting the COVID-19 pandemic. Front. Immunol. 12, 637651. https://doi.org/10.3389/fimmu.2021.637651.
Shrock, E., Fujimura, E., Kula, T., Timms, R.T., Lee, I.H., Leng, Y., Robinson, M.L., Sie, B.M., Li, M.Z., Chen, Y., et al.; MGHCOVID-19 Collection & Processing Team (2020). Viral epitope profiling of COVID-19 patients reveals crossreactivity and correlates of severity. Science 370, eabd4250. https://doi.org/10.1126/science.abd4250.
Smatti, M.K., Al Thani, A.A., and Yassine, H.M. (2018). Viral-induced enhanced disease illness. Front. Microbiol. 9, 2991. https://doi.org/10.3389/fmich.2018. 02991.
Tang, Y., Liu, J., Zhang, D., Xu, Z., Ji, J., and Wen, C. (2020). Cytokine storm in COVID-19: the current evidence and treatment strategies. Front. Immunol. 11. https://doi.org/10.3389/fimmu.2020.01708.

Thulin, N.K., Brewer, R.C., Sherwood, R., Bournazos, S., Edwards, K.G., Ramadoss, N.S., Taubenberger, J.K., Memoli, M., Gentles, A.J., Jagannathan, P., et al. (2020). Maternal anti-dengue IgG fucosylation predicts susceptibility to dengue disease in infants. Cell Rep. 31, 107642. https://doi.org/10.1016/j.celrep. 2020.107642.
Tillett, R.L., Sevinsky, J.R., Hartley, P.D., Kerwin, H., Crawford, N., Gorzalski, A., Laverdure, C., Verma, S.C., Rossetto, C.C., Jackson, D., et al. (2021). Genomic evidence for reinfection with SARS-CoV-2: a case study. Lancet Infect. Dis. 21, 52-58. https://doi.org/10.1016/S1473-3099 (20) 30764-7.
Tripet, B., Kao, D.J., Jeffers, S.A., Holmes, K.V., and Hodges, R.S. (2006). Template-based coiled-coil antigens elicit neutralizing antibodies to the SARS-coronavirus. J. Struct. Biol. 155, 176-194. https://doi.org/10.1016/j. jsb.2006.03.019.
Unterman, A., Sumida, T.S., Nouri, N., Yan, X., Zhao, A.Y., Gasque, V., Schupp, J.C., Asashima, H., Liu, Y., Cosme, C., Jr., et al.; The Yale IMPACT Research Team (2022). Single-cell multi-omics reveals dyssynchrony of the innate and adaptive immune system in progressive COVID-19. Nat. Commun. 13, 440. https://doi.org/10.1038/s41467-021-27716-4.
Vogelpoel, L.T.C., Baeten, D.L.P., de Jong, E.C., and den Dunnen, J. (2015). Control of cytokine production by human fc gamma receptors: implications for pathogen defense and autoimmunity. Front. Immunol. 6, 79. https://doi. org/10.3389/fimmu.2015.00079.
Walls, A.C., Tortorici, M.A., Snijder, J., Xiong, X., Bosch, B.J., Rey, F.A., and Veesler, D. (2017). Tectonic conformational changes of a coronavirus spike glycoprotein promote membrane fusion. Proc. Natl. Acad. Sci. U S A. 114, 11157-11162. https://doi.org/10.1073/pnas. 1708727114.
Wang, H., Wu, X., Zhang, X., Hou, X., Liang, T., Wang, D., Teng, F., Dai, J., Duan, H., Guo, S., et al. (2020a). SARS-CoV-2 proteome microarray for mapping COVID-19 antibody interactions at amino acid resolution. ACS Cent. Sci. 6, 2238-2249. https://doi.org/10.1021/acscentsci.0c00742.
Wang, Q., Zhang, Y., Wu, L., Niu, S., Song, C., Zhang, Z., Lu, G., Qiao, C., Hu, Y., Yuen, K.Y., et al. (2020b). Structural and functional basis of SARS-CoV-2 entry by using human ACE2. Cell 181, 894-904.e9. https://doi.org/10.1016/j. cell.2020.03.045.
Wang, S.F., Tseng, S.P., Yen, C.H., Yang, J.Y., Tsao, C.H., Shen, C.W., Chen, K.H., Liu, F.T., Liu, W.T., Chen, Y.M.A., and Huang, J.C. (2014). Antibodydependent SARS coronavirus infection is mediated by antibodies against spike proteins. Biochem. Biophys. Res. Commun. 451, 208-214. https://doi.org/10.1016/j.bbrc.2014.07.090.
Wang, T.T., Sewatanon, J., Memoli, M.J., Wrammert, J., Bournazos, S., Bhaumik, S.K., Pinsky, B.A., Chokephaibulkit, K., Onlamoon, N., Pattanapanyasat, K., et al. (2017). IgG antibodies to dengue enhanced for FcgRIIIA binding determine disease severity. Science 355, 395-398. https://doi.org/10.1126/science. aai8128.
Wang, Y., Zhang, L., Sang, L., Ye, F., Ruan, S., Zhong, B., Song, T., Alshukairi, A.N., Chen, R., Zhang, Z., et al. (2020c). Kinetics of viral load and antibody response in relation to COVID-19 severity. J. Clin. Investig. 130, 5235-5244. https://doi.org/10.1172/JCI138759.
Wec, A.Z., Wrapp, D., Herbert, A.S., Maurer, D.P., Haslwanter, D., Sakharkar, M., Jangra, R.K., Dieterle, M.E., Lilov, A., Huang, D., et al. (2020). Broad neutralization of SARS-related viruses by human monoclonal antibodies. Science 369, 731-736. https://doi.org/10.1126/science.abc7424.
Wu, Z., and McGoogan, J.M. (2020). Characteristics of and important lessons from the coronavirus disease 2019 (COVID-19) outbreak in China. JAMA 323, 1239-1242. https://doi.org/10.1001/jama.2020.2648.
Xia, S., Liu, M., Wang, C., Xu, W., Lan, Q., Feng, S., Qi, F., Bao, L., Du, L., Liu, S., et al. (2020). Inhibition of SARS-CoV-2 (previously 2019-nCoV) infection by a highly potent pan-coronavirus fusion inhibitor targeting its spike protein that harbors a high capacity to mediate membrane fusion. Cell Res. 30, 343-355. https://doi.org/10.1038/s41422-020-0305-x.
Ye, Q., Wang, B., and Mao, J. (2020). The pathogenesis and treatment of the ‘Cytokine Storm’ in COVID-19. J. Infect. 80, 607-613. https://doi.org/10.1016/j.jinf.2020.03.037.
Yip, M.S., Leung, N.H.L., Cheung, C.Y., Li, P.H., Lee, H.H.Y., Daeron, M., Peiris, J.S.M., Bruzzone, R., and Jaume, M. (2014). Antibody-dependent infection of human macrophages by severe acute respiratory syndrome coronavirus. Virol. J. 11, 82. https://doi.org/10.1186/1743-422X-11-82.
Young, B.E., Ong, S.W.X., Ng, L.F.P., Anderson, D.E., Chia, W.N., Chia, P.Y., Ang, L.W., Mak, T.M., Kalimuddin, S., Chai, L.Y.A., et al.; Singapore 2019 Novel Coronavirus Outbreak Research Team (2020). Viral dynamics and immune correlates of coronavirus disease 2019 (COVID-19) severity. Clin. Infect. Dis. 73, e2932-e2942. https://doi.org/10.1093/cid/ciaa1280.
Zamecnik, C.R., Rajan, J.V., Yamauchi, K.A., Mann, S.A., Loudermilk, R.P., Sowa, G.M., Zorn, K.C., Alvarenga, B.D., Gaebler, C., Caskey, M., et al. (2020). ReScan, a multiplex diagnostic pipeline, pans human sera for SARSCoV-2 antigens. Cell Rep. Med. 1, 100123. https://doi.org/10.1016/j.xcrm. 2020.100123.
Zhang, B., Zhou, X., Zhu, C., Song, Y., Feng, F., Qiu, Y., Feng, J., Jia, Q., Song, Q., Zhu, B., and Wang, J. (2020). Immune phenotyping based on the neutrophil-to-lymphocyte ratio and IgG level predicts disease severity and outcome for patients with COVID-19. Front. Mol. Biosci. 7, 157. https://doi.org/10.3389/fmolb.2020.00157.
Zhao, J., Yuan, Q., Wang, H., Liu, W., Liao, X., Su, Y., Wang, X., Yuan, J., Li, T., Li, J., et al. (2020). Antibody responses to SARS-CoV-2 in patients with novel coronavirus disease 2019. Clin. Infect. Dis. 71, 2027-2034. https://doi.org/10. 1093/cid/ciaa344.
Zost, S.J., Gilchuk, P., Chen, R.E., Case, J.B., Reidy, J.X., Trivette, A., Nargi, R.S., Sutton, R.E., Suryadevara, N., Chen, E.C., et al. (2020). Rapid isolation and profiling of a diverse panel of human monoclonal antibodies targeting the SARS-CoV-2 spike protein. Nat. Med. 26, 1422-1427. https://doi.org/10. 1038/s41591-020-0998-x.

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A substrate or plurality of substrates comprising at least one peptide selected from a plurality of peptides attached to said substrate or plurality of substrates, the plurality of peptides selected from the group consisting of:
i) a betacoronavirus (“BC”) spike protein receptor binding domain (RBD) sequence;
ii) a first BC spike protein C-terminus domain (CTD1) sequence that is optionally pep4;
iii) a second BC spike protein C-terminus domain (CTD2) sequence that is optionally pep1;
iv) a BC spike protein Furin cleavage site (S1/S2) sequence that is optionally pep2;
v) a BC spike protein S2′ Fusion protein (S2′FP) sequence that is optionally pep5;
vi) a BC spike protein 5′ flanking Heptad Repeat-2 (5′fHR2) that is optionally pep6;
vii) a BC spike protein Heptad Repeat-2 (HR2) that is optionally pep3; and combinations thereof.

2. The plurality of substrates of claim 1, comprising 2, 3, 4, 5, 6, or 7 substrates, each substrate comprising only one of the peptides, and each substrate comprising a different peptide attached to said substrates.

3. The plurality of substrates of claim 2, wherein at least one of the peptides is modified to include at least one linker amino acid sequence.

4. The plurality of substrates of claim 2, wherein the plurality of peptides comprises at least two of the RBD, pep3, pep5, and pep6.

5. The plurality of substrates of claim 2, wherein the plurality of peptides comprises at least the pep3 and the pep5.

6. The plurality of substrates of claim 2, wherein the plurality of peptides comprises at least the RBD and the pep5.

7. The plurality of substrates of claim 2, wherein the plurality of peptides comprises at least the pep5 and the pep6.

8. The plurality of substrates of claim 2, wherein the plurality of peptides comprises at least the pep3 and the RBD.

9. The plurality of substrates of claim 2, wherein the plurality of peptides comprises at least the pep3, the RBD and the pep5.

10. The plurality of substrates of claim 2, wherein the plurality of peptides comprises at least the pep3 and the pep6.

11. The plurality of substrates of claim 2, wherein the substrate is in contact with a sample obtained from an individual.

12. The plurality of substrates of claim 11, wherein the individual has not been exposed to SARS-CoV-2 before the sample was obtained.

13. The plurality of substrates of claim 11, wherein the individual has been exposed to SARS-CoV-2 before the sample was obtained.

14. The plurality of substrates of claim 11, wherein the individual developed COVID-19 before the sample was obtained.

16. The plurality of substrates of claim 11, wherein the individual has not been vaccinated against SARS-CoV-2.

17. The plurality of substrates of claim 11, wherein the individual has been vaccinated against SARS-CoV-2.

18. The plurality of substrates of any one of claims 3-10, further comprising antibodies produced by the individual, wherein at least some of the antibodies produced by the individual are bound to at least some of the peptides.

19. The plurality of substrates of claim 18, further comprising detectably labeled antibodies bound to the antibodies produced by the individual, wherein the antibodies produced by the individual are also bound to at least some of the peptides.

20. A method comprising contacting a biological sample obtained from an individual with a plurality of substrates of any one of claims 2-10.

21. The method of claim 20, comprising determining antibodies bound to said plurality of substrates.

22. The method of claim 21, wherein the determining antibodies comprises determining a ratio of antibodies bound to at least one of the peptides relative to antibodies bound to at least one other of the peptides.

23. The method of claim 21, wherein the ratio comprises at least one of:
a) antibodies bound to pep3 relative to antibodies bound to pep5;
b) antibodies bound to RBD relative to antibodies bound to pep5;
c) antibodies bound to pep5 relative to antibodies bound to pep6;
d) antibodies bound to pep3 relative to antibodies bound to RBD;
e) antibodies bound to a combination of pep3 and RBD relative to antibodies bound to pep5; or
f) antibodies bound to a combination of pep3 and pep6 relative to antibodies to pep5.

24. The method of claim 23, wherein the plurality of substrates are present in enzyme-linked immunosorbent assays (ELISAs).

25. The method of claim 24, wherein the individual has not been exposed to SARS-CoV-2 before the sample was obtained.

26. The method of claim 24, wherein the individual has been exposed to SARS-CoV-2 before the sample was obtained.

27. The method of claim 24, wherein the individual developed COVID-19 before the sample was obtained.

28. The method of claim 24, wherein the individual has not been vaccinated against SARS-CoV-2.

29. The method of claim 24, wherein the individual has been vaccinated against SARS-CoV-2.

30. The method of claim 24, further comprising, based on the antibody determination, that the individual is in need of therapy for SARS-CoV-2 infection, the method further comprising administering to the individual an anti-viral agent, wherein optionally, the therapeutic agent is selected based at least in part on the antibody determination.

31. The method of claim 24, further comprising, based on the antibody determination, that the individual is in need of a vaccine against SARS-CoV-2, the method further comprising administering the vaccine to the individual, wherein optionally, the vaccine is selected based at least in part on the antibody determination.

32. The method of claim 24, wherein the individual has been vaccinated prior to obtaining the sample, the method comprising administering to the individual a subsequent vaccine of the same type as the vaccine previously administered to the individual, or administering a different vaccine to the individual, wherein optionally the vaccine is selected based in part on the antibody determination.

35. The method of claim 24, comprising determining if the individual will have a protective or inefficient or deleterious antibody response from exposure to SARS-CoV-2.

36. The method of claim 24, comprising determining risk of severe COVID-19 for the individual based on the antibody determination.

37. A pharmaceutical formulation comprising one or a plurality of peptides as in any one of claims 1-10, the pharmaceutical formulation optionally comprising an adjuvant.

38. A kit comprising a plurality of peptides as in any one of claims 1-10, the kit optionally further comprising one or more reagents for use in determining antibodies from a biological sample.

39. A method comprising detecting a signal from detectably labeled antibodies from claim 19.

40. A system configured to detect the signal of claim 39, the system comprising a processor configured to run software to analyze the antibody determination.
